(19) United States
(12) Patent Application Publication
(10) Pub. No.: US 2026/0263698 A1
(43) Pub. Date: Sep. 10, 2026

(54) SYSTEM AND METHOD FOR INJECTION

(71) Applicant: CREDENCE MEDSYSTEMS, INC., Menlo Park, CA (US)

(72) Inventor: Alan E. Shluzas, San Carlos, CA (US)

(73) Assignee: CREDENCE MEDSYSTEMS, INC., Menlo Park, CA (US)

(21) Appl. No.: 19/558,261

(22) Filed: Mar. 5, 2026

Related U.S. Application Data
(60) Provisional application No. 63/767,424, filed on Mar. 5, 2025.

(51) Int. Cl. A61M 5/31; A61M 5/315

(52) U.S. Cl. CPC A61M 5/3137; A61M 5/31501; A61M 5/31515; A61M 5/31535; A61M 5/31573; A61M 5/31578; A61M 2005/3139; A61M 2005/31508; A61M 2202/0007; A61M 2202/04; A61M 2205/581; A61M 2205/582

(57) ABSTRACT

A system for injecting includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a thumbpad at a proximal end thereof, and a distal end configured to be disposed adjacent to the stopper member. In addition, the system includes a finger flange removably coupled to the syringe flange. The system also includes a collar rotatably coupled to the finger flange and disposed around the plunger member to control movement of the plunger member relative to the finger flange.

The present application claims priority to U.S. Provisional Patent Application Serial Number 63/767,424, filed Mar. 5, 2025 under attorney docket number CM.30050.00, and entitled "SYSTEM AND METHOD FOR INJECTION". This application includes subject matter similar to the subject matter described in the following co-owned U.S. patent applications: (1) U.S. Utility Patent Application Serial Number 16/683,126, filed on Nov. 13, 2019 under attorney docket number CM.20022-1.00, and entitled "SYSTEM AND METHOD FOR MICRODOSE INJECTION"; (2) U.S. Utility Patent Application Serial Number 14/696,342, filed Apr. 24, 2015 under attorney docket number CM.20003.00, and entitled "SYSTEM AND METHOD FOR SAFETY SYRINGE"; (3) U.S. Utility Patent Application Serial Number 14/543,787, filed Nov. 17, 2014 under attorney docket number CM.20002.00, and entitled "SYSTEM AND METHOD FOR DRUG DELIVERY WITH A SAFETY SYRINGE"; (4) U.S. Utility Patent Application Serial Number 14/321,706, filed Jul. 1, 2014 under attorney docket number CM.20001.00, and entitled "SAFETY SYRINGE"; (5) U.S. Utility Patent Application Serial Number 15/801,239, filed Nov. 1, 2017 under attorney docket number CM.20011.00, and entitled "SYSTEM AND METHOD FOR SAFETY SYRINGE";

(6) U.S. Utility Patent Application Serial Number 15/801,259, filed Nov. 1, 2017 under attorney docket number CM.20012.00, and entitled "SYSTEM AND METHOD FOR SAFETY SYRINGE"; (7) U.S. Utility Patent Application Serial Number 15/801,304, filed Nov. 1, 2017 under attorney docket number CM.20015.00, and entitled "SYSTEM AND METHOD FOR SAFETY SYRINGE;

(8) U.S. Utility Patent Application Serial Number 15/985,354, filed May 21, 2018 under attorney docket number CM.20016.00, and entitled "SYSTEM AND METHOD FOR COLLECTING INJECTION INFORMATION"; (9) U.S. Utility Patent Application Serial Number 62/542,230, filed Aug. 7, 2017 under attorney docket number CM.00.30013, and entitled "CARTRIDGE SAFETY INJECTION SYSTEM AND METHODS"; (10) U.S. Utility Patent Application Serial Number 15/801,239, filed Nov. 1, 2017 under attorney docket number CM.20011.00, and entitled "SYSTEM AND METHOD FOR SAFETY SYRINGE"; (11) U.S. Utility Patent Application Serial Number 15/801,259, filed Nov. 1, 2017 under attorney docket number CM.20012.00, and entitled "SYSTEM AND METHOD FOR SAFETY SYRINGE"; (12) U.S. Utility Patent Application Serial Number 15/801,281, filed Nov. 1, 2017 under attorney docket number CM.20013.00, and entitled "CARTRIDGE SAFETY INJECTION SYSTEM AND METHODS"; (13) U.S. Utility Patent Application Serial Number 15/801,304, filed Nov. 1, 2017 under attorney docket number CM.20015.00, and entitled "SYSTEM AND METHOD FOR SAFETY SYRINGE"; (14) U.S. Utility Patent Application Serial Number 16/011,453, filed Jun. 18, 2018 under attorney docket number CM.20014.00, and entitled "SYSTEM AND METHOD FOR SAFETY SYRINGE";

(15) U.S. Utility Patent Application Serial Number 15/985,354, filed May 21, 2018 under attorney docket number CM.20016.00, and entitled "SYSTEM AND METHOD FOR COLLECTING INJECTION INFORMATION"; (16) U.S. Utility Patent Application Serial Number 16/683,157, filed Nov. 13, 2019 under attorney docket number CM.20022-2.00, and entitled "SYSTEM AND METHOD

FOR MULTIPLE SITE INJECTION"; (17) U.S. Utility Patent Application Serial Number 17/351,006, filed Jun. 17, 2021 under attorney docket number CM.20029.00, and entitled "SYSTEM AND METHOD FOR MICRODOSE INJECTION"; (18) U.S. Provisional Patent Application Serial Number 63/758,909, filed Feb. 14, 2025 under attorney docket number CM.30049.00, and entitled "SYSTEM AND METHOD FOR INJECTION"; and (19) U.S. Utility Patent Application Serial Number 19/540,044, filed Feb. 13, 2026 under attorney docket number CM.20049.00, and entitled "SYSTEM AND METHOD FOR INJECTION." The contents of the applications identified herein are fully incorporated herein by reference as though set forth in full.

### FIELD OF THE INVENTION

The present invention relates generally to injection systems, devices, and processes for facilitating various levels of control over fluid infusion, and more particularly to systems and methods related to syringes for delivery microliter range doses of fluids in healthcare environments.

### BACKGROUND

Millions of syringes 2, such as that depicted in FIG. 1A, are consumed in healthcare environments every day. A typical syringe 2 includes a tubular body 4, a plunger 6, and an injection needle 8. As shown in FIG. 1B, such a syringe 2 may be utilized not only to inject fluid into a patient, but also to withdraw or expel fluid out of or into a container such as a medicine bottle, vial, bag, or other drug containment system 10. Indeed, due to regulatory constraints in some countries such as the United States as well as sterility maintenance concerns, upon use of a medicine bottle 10 with a syringe 2 as shown in a particular patient's environment, such medicine bottle may only be utilized with a single patient and then must be disposed of - causing significant medical waste from bottle and remaining medicine disposal, and even contributing to periodic shortages of certain critical drugs.

Referring to FIG. 2A, three Luer-type syringes 12 are depicted, each having a Luer fitting geometry 14 disposed distally, so that they may be coupled with other devices having similar mating geometry, such as the Luer manifold assembly 16 depicted in FIG. 2B. The Luer manifold assembly of FIG. 2B may be used to administer liquid drugs to the patient intravenously with or without the use of an intravenous infusion bag. The Luer fittings 14 of the syringes of FIG. 2A may be termed the "male" Luer fittings, while those of FIG. 2B 18 may be termed the "female" Luer fittings; one of the Luer interfaces may be threaded (in which case the configuration may be referred to as a "Luer lock" configuration) so that the two sides may be coupled by relative rotation, which may be combined with compressive loading. In other words, in one Luer lock embodiment, rotation, possibly along with compression, may be utilized to engage threads within the male fitting 14 which are configured to engage a flange on the female fitting 18 and bring the devices together into a fluid-sealed coupling. In another embodiment, tapered interfacing geometries may be utilized to provide for a Luer engagement using compression without threads or rotation (such a configuration may be referred to as a "slip-on" or "conical" Luer configuration). While such Luer couplings are perceived to be relatively safe for operators, there is risk of medicine spilling/leaking and parts breakage during the loading to provide a Luer coupling. The use of needle injection configurations, on the other hand, carries with it the risk of a sharp needle contacting or poking a person or structure that is not desired. For this reason, so called "safety syringes" have been developed.

One embodiment of a safety syringe 20 is shown in FIG. 3, wherein a tubular shield member 22 is spring biased to cover the needle 8 when released from a locked position relative to the syringe body 4. Another embodiment of a safety syringe 24 is shown in FIGS. 4A-4B. With such a configuration, after full insertion of the plunger 6 relative to the syringe body 4, the retractable needle 26 is configured to retract 28, 26 back to a safe position within the tubular body 4, as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for pre-filled syringe assemblies such as those depicted in FIGS. 5A and 5B, which generally include a syringe body, or "drug enclosure containment delivery system", 34, a plunger tip, plug, or stopper 36, and a distal seal or cap 35 which may be fitted over a Luer type interface (FIG. 5A shows the cap 35 in place; FIG. 5B has the cap removed to illustrate the Luer interface 14. Liquid medicine may reside in the volume, or medicine reservoir, 40 between the distal seal 35 and the distal end 37 of the stopper member 36. The stopper member 36 may include a standard butyl rubber material and may be coated, such as with a biocompatible lubricious coating (e.g., polytetrafluoroethylene ("PTFE")), to facilitate preferred sealing and relative motion characteristics against the associated syringe body 34 structure and material. The proximal end of the syringe body 34 in FIG. 5B includes a conventional integral syringe flange 38), which is formed integral to the material of the syringe body 34. The flange 38 is configured to extend radially from the syringe body 34 and may be configured to be a full circumference, or a partial circumference around the syringe body 34. A partial flange is known as a "clipped flange" while the other is known as a "full flange." The flange is used to grasp the syringe with the fingers to provide support for pushing on the plunger to give the injection. The syringe body 34 preferably includes a translucent material such as a glass or polymer. To form a contained volume within the medicine chamber or reservoir 40, and to assist with expulsion of the associated fluid through the needle, a stopper member 36 may be positioned within the syringe body 34. The syringe body may define a substantially cylindrical shape (i.e., so that a plunger tip 36 having a circular cross sectional shape may establish a seal against the syringe body), or be configured to have other cross sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more "vertical" solutions, such as that 41 featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Moreover, injection systems have reduced accuracy and precision as the volume of injectable fluid is reduced into the microliter range. In particular, removing air from the syringe body ("de-bubbling") before injection is difficult to perform accurately and precisely for such injection systems. There is a need for injection systems which address shortcomings of currently-available configurations. In particular, there is a need for injection systems that perform (e.g., de-bubble and inject) accurately in the microliter range. It is also desirable that such syringe assemblies may utilize the existing and relatively well-controlled supply chain of conventionally delivered pre-filled syringe bodies and other off-the-shelf components, and the corresponding assembly machinery and personnel.

### SUMMARY

Embodiments are directed to injection systems. In particular, the embodiments are directed to microliter range injection systems that include at least some off-the-shelf syringe components.

In one embodiment, a system for injecting includes a syringe body having proximal and distal ends, a syringe interior, and a syringe flange at the proximal end thereof. The system also includes an injectable fluid disposed in the syringe interior. The system further includes a stopper member disposed in the syringe interior. Moreover, the system includes a plunger member having a thumbpad at a proximal end thereof, and a distal end configured to be disposed adjacent to the stopper member. In addition, the system includes a finger flange removably coupled to the syringe flange. The system also includes a collar rotatably coupled to the finger flange and disposed around the plunger member to control movement of the plunger member relative to the finger flange.

In one or more embodiments, the plunger member is keyed to the finger flange to prevent rotation of the plunger member relative to the finger flange. The distal end of the plunger member may be coupled to the stopper member. The collar may include a proximally facing stop surface defining a central opening. The plunger member may include a pair of externally extending ribs configured to cooperate with the proximally facing stop surface and the central opening to control longitudinal movements of the plunger member relative to the finger flange. The collar may be disposed longitudinally between the thumbpad and the finger flange. In one or more embodiments, the finger flange includes a proximally extending member having a pair of flex arms configured to provide an audible and/or tactile cue during rotation of the collar about the proximally extending member. The finger flange also includes a rotational stop rib configured to limit rotation of the collar about the proximally extending member. The collar may include a pair of proximally facing proximal arcuate stops that define a corresponding pair of proximal longitudinal slots, and a pair of proximally facing distal arcuate stops that define a corresponding pair of distal longitudinal slots. The proximally facing proximal arcuate stops and the proximally facing distal arcuate stops may be rotationally displaced from each other.

In one or more embodiments, the plunger member includes proximal and distal pairs of externally extending ribs configured to cooperate with the proximally facing proximal and distal arcuate stops to control longitudinal movements of the plunger member relative to the finger flange. The distal pair of externally extending ribs may be configured to control rotation of the collar. The proximal and distal pairs of externally extending ribs may define a respective pair of longitudinal spaces therebetween.

In one or more embodiments, the system has a transport configuration in which the plunger member is in a proximal most position, a primed/injection ready configuration in which the plunger member is in an intermediate position, an injected configuration in which the plunger member is in a distal most position, a first stage in which the system can convert from the transport configuration to the primed/injection ready configuration, and a second stage in which the system can convert from the primed/injection ready configuration to the injected configuration. The finger flange may define a pointer. The collar may define a first external indicia of the first stage and a second external indicia of the second stage. The pointer on the finger flange may be configured to align with the first external indicia on the collar when the system is in the first stage and align with the second external indicia on the collar when the system is in the second stage.

In one or more embodiments, the system is configured to transition from the first stage to the second stage by rotation of the collar relative to the finger flange. In the injected configuration, less than a total amount of the injectable fluid may be ejected from the syringe interior. The collar may define an external indicia of rotation direction. The plunger rod may include a barb configured to interact with a rib on the finger flange to limit proximal movement of the plunger rod relative to the finger flange.

In one or more embodiments, the collar includes a distally extending member having an external indicia of system mode defined thereon. The finger flange includes a proximally extending member defining a viewing window configured to align with and display the indicia of system mode defined on the distally extending member of the collar. The distally extending member of the collar is configured to be at least partially disposed coaxially within the proximally extending member of the finger flange. The indicia of system mode and the viewing window may be configured to indicate a prime mode and a dose mode of the system.

In one or more embodiments, the distally extending member of the collar defines an axially extending rib, and the proximally extend member of the finger flange defines a detent rib and a rotational stop rib. The axially extending rib may be configured to cooperate with the detent rib to generate an audible/tactile click during a mode switch. The axially extending rib may be configured to cooperate with the rotational stop rib to limit rotation of the collar relative to the finger flange.

In another embodiment, a method for injecting a fluid includes providing a syringe assembly including a syringe body having proximal and distal ends, a syringe interior, and a syringe flange at the proximal end thereof. The syringe assembly also includes an injectable fluid disposed in the syringe interior. The syringe assembly further includes a stopper member disposed in the syringe interior. Moreover, the syringe assembly includes a plunger member having a thumbpad at a proximal end thereof, and a distal end configured to be disposed adjacent to the stopper member. In addition, the syringe assembly includes a finger flange removably coupled to the syringe flange. The syringe assembly also includes a collar rotatably coupled to the finger flange and disposed around the plunger member to control movement of the plunger member relative to the finger flange. The syringe assembly has a first stage configured for transport and purging of air in the syringe interior, and a second stage configured for delivery of a portion of the injectable fluid in the syringe interior. The method also includes with the syringe assembly in the first stage, applying a first distally directed force to the thumbpad to advance the plunger member and the stopper member to a first distal end position to expel air from the syringe interior through the distal end of the syringe body.

The method further includes rotating the collar relative to the finger flange to a rotation end location to transform the syringe assembly from the first stage to the second stage. Moreover, the method includes with the syringe assembly in the first stage, applying a second distally directed force to the thumbpad to advance the plunger member and the stopper member to a second distal end position to expel the portion of the injectable fluid from the syringe interior through the distal end of the syringe body.

in one or more embodiments, the syringe assembly has a transport configuration in which the plunger member is in a proximal most position, a primed/injection ready configuration in which the plunger member is in the first distal end position, and an injected configuration in which the plunger member is in the second distal end position. Applying the first distally directed force to the thumbpad to advance the plunger member and the stopper member to the first distal end position and rotating the collar relative to the finger flange to the rotation end location may transform the syringe assembly from the transport configuration to the primed/injection ready configuration. Applying the first distally directed force to the thumbpad to advance the plunger member and the stopper member to the first distal end position may transform the syringe assembly from the primed/injection ready configuration to the injected configuration. The collar may define a first external indicia of the first stage and a second external indicia of the second stage. The finger flange may define a pointer. Rotating the collar relative to the finger flange to the rotation end location to transform the system to the primed/injection ready configuration may move the pointer on the finger flange from the first external indicia to the second external indicia on the finger flange.

In one or more embodiments, the finger flange includes a proximally extending member having a pair of flex arms. The pair of flex arms may provide an audible and/or tactile cue during rotation of the collar about the proximallyextending member. The plunger rod may include a barb. The bard may interact with a rib on the finger flange to limit proximal movement of the plunger rod relative to the finger flange. In the injected configuration, the portion of the injectable fluid ejected from the syringe interior may be less than a total amount of the injectable fluid in the syringe interior.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

The foregoing and other aspects of embodiments are described in further detail with reference to the accompanying drawings, in which the same elements in different figures are referred to by common reference numerals, wherein:

FIGS. 1A-5C illustrate various aspects of conventional injection syringe configurations.

FIG. 6 and 7 are side and exploded views depicting systems for injecting according to some embodiments.

FIGS. 8A and 8B are longitudinal cross-sectional and side views depicting a system for injecting in a transport configuration according to some embodiments.

FIGS. 9A and 9B are longitudinal cross-sectional and side views depicting a system for injecting in a primed configuration according to some embodiments.

FIGS. 10A and 10B are longitudinal cross-sectional and side views depicting a system for injecting in an injection ready configuration according to some embodiments.

FIGS. 11A and 11B are longitudinal cross-sectional and side views depicting a system for injecting in an injected configuration according to some embodiments.

FIG. 12 is a detailed perspective view depicting a system for injecting in a primed configuration according to some embodiments, with the collar shown in shadow.

FIG. 13 is a detailed perspective view depicting a system for injecting in a primed configuration according to some embodiments, with the collar omitted for clarity.

FIG. 14 is a detailed perspective view depicting a system for injecting in a primed configuration according to some embodiments, with the syringe body and stopper member omitted for clarity.

FIGS. 15 and 16 are proximal and distal perspective views depicting a collar for use with systems for injecting according to some embodiments.

FIG. 17 is a longitudinal cross-sectional view depicting a collar for use with systems for injecting according to some embodiments.

FIGS. 18, 19, and 20 are side, bottom, and top views depicting a finger flange for use with systems for injecting according to some embodiments.

FIGS. 21A and 21B are side and perspective views depicting a plunger member for use with systems for injecting according to some embodiments.

FIG. 22 is a perspective view depicting a plunger member for use with systems for injecting according to some embodiments.

FIGS. 23A and 23B are side and longitudinal cross-sectional views depicting a system for injecting in an injection ready configuration according to some embodiments.

FIGS. 24, 25, and 26 are side, longitudinal cross-sectional, and exploded views depicting systems for injecting according to some embodiments.

FIGS. 27 and 28 are perspective and top views depicting a finger flange for use with systems for injecting according to some embodiments.

FIGS. 29A and 29B are proximal and distal perspective views depicting a collar for use with systems for injecting according to some embodiments.

FIG. 30 is a perspective view depicting a system for injecting in a transport configuration according to some embodiments.

FIG. 31 is a perspective view depicting a system for injecting in a primed configuration according to some embodiments.

FIG. 32 is a perspective view depicting a system for injecting in an injection ready configuration according to some embodiments.

FIG. 33 is a perspective view depicting a system for injecting in an injected configuration according to some embodiments. In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

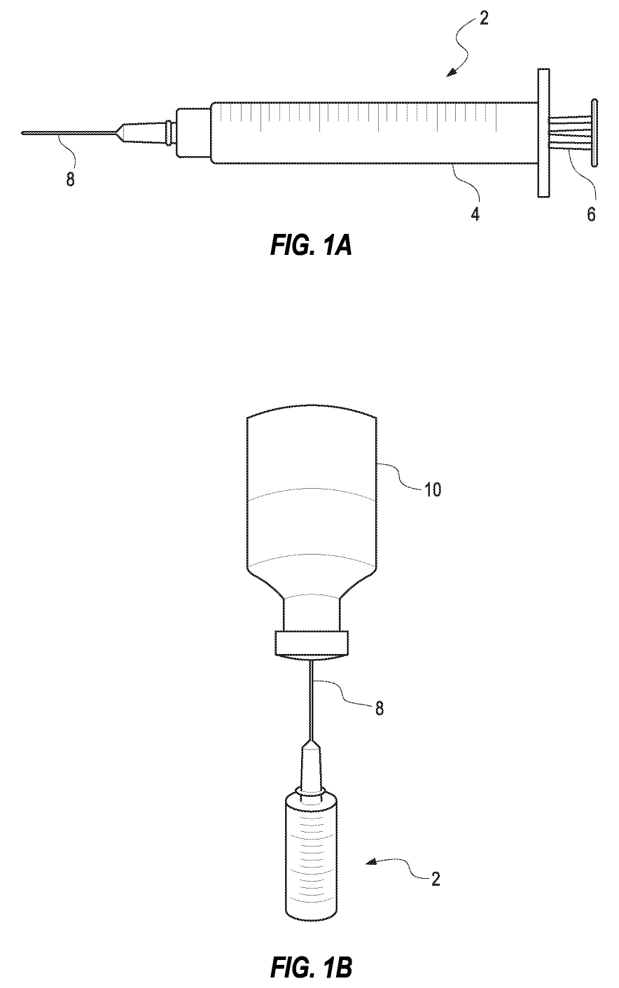

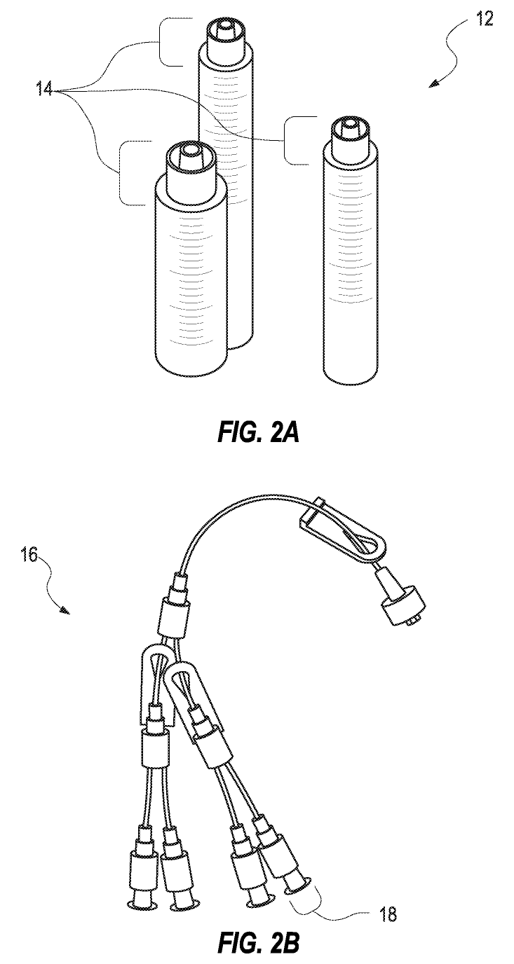

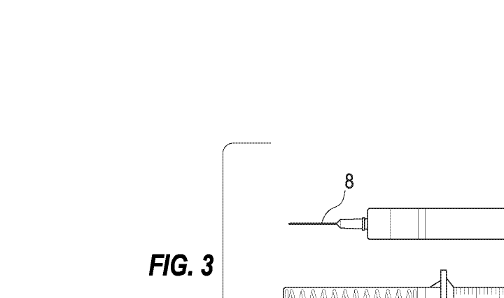

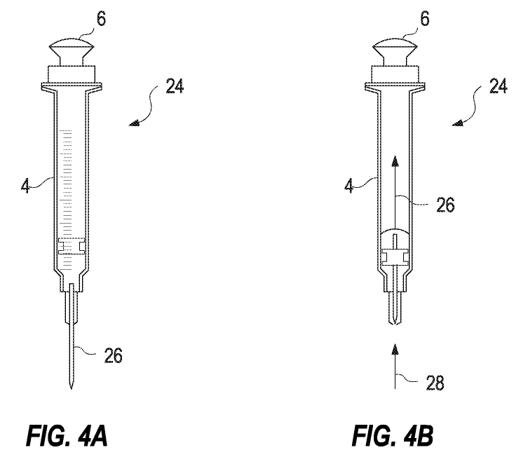

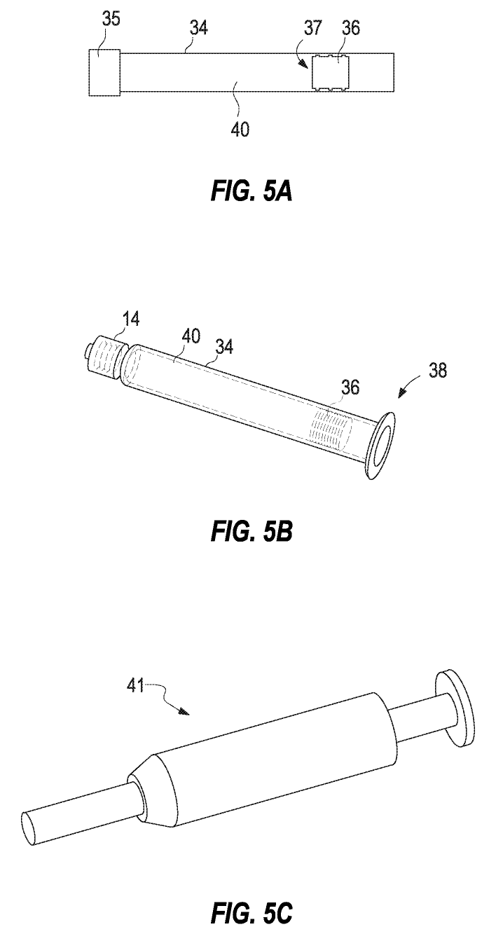

### DETAILED DESCRIPTION OF ILLUSTRATED EMBODIMENTS

### Exemplary Injection Systems

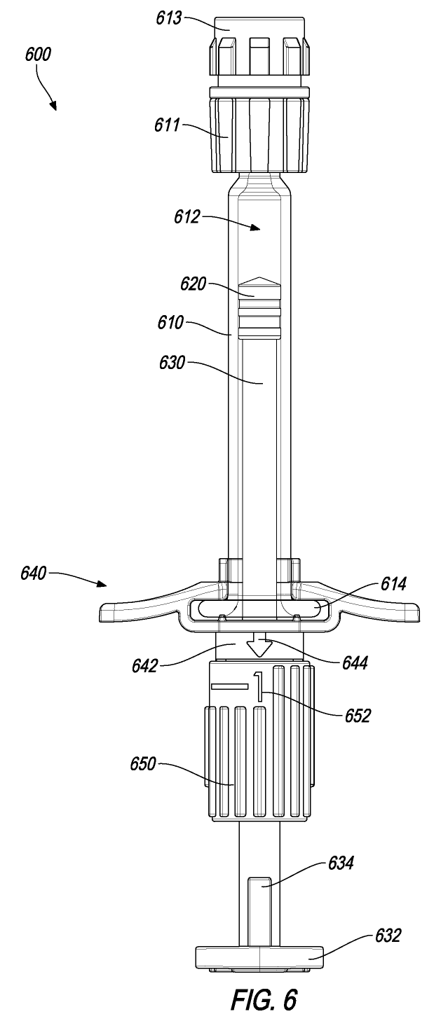

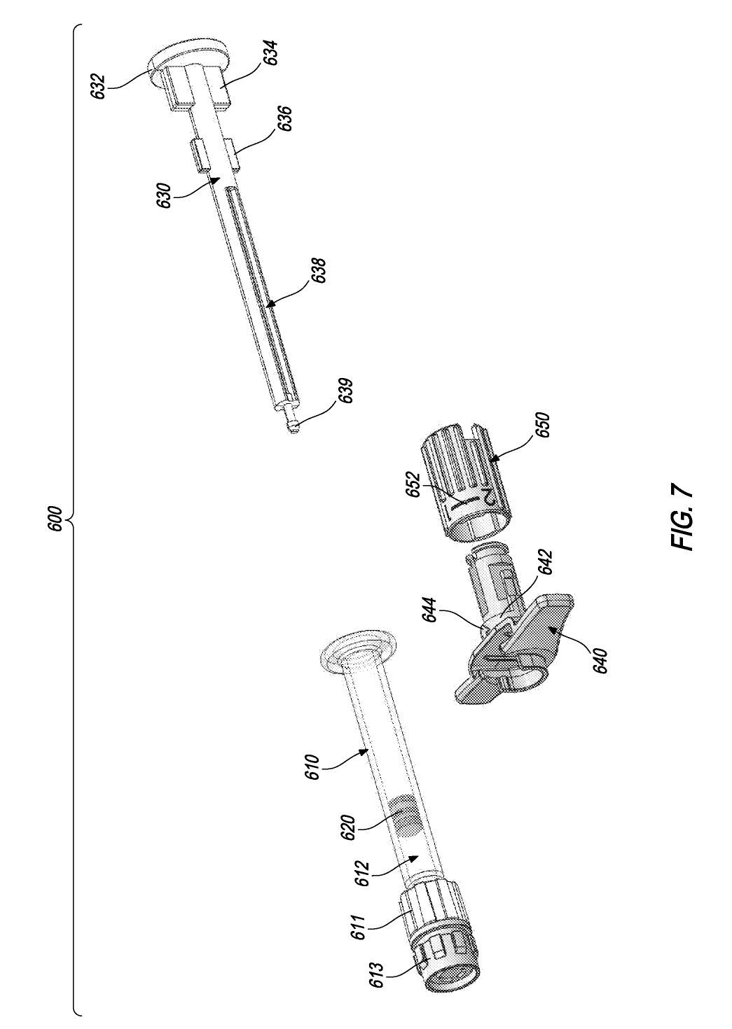

FIG. 6 and 7 are side and exploded views depicting systems for injecting according to some embodiments. The system 600 includes a syringe body 610 having proximal and distal ends, a syringe interior 612, and a syringe flange 614 at the proximal end thereof. A stopper member 620 is disposed in the syringe interior 612 to form a fluid chamber therein. The syringe interior/fluid chamber 612 contains an injectable fluid (not shown) and some air. A plunger member 630 is partially disposed in the syringe interior 612 and coupled to the stopper member 620. The system 600 also includes a finger flange 640 coupled to the syringe flange 612. The system 600 further includes a dose setting collar 650 coupled to the finger flange 640 and the plunger member 630.

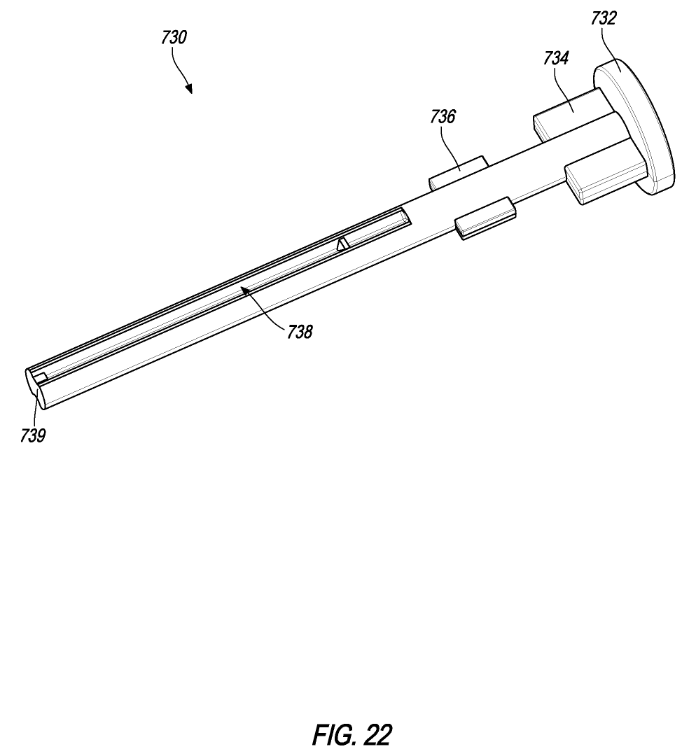

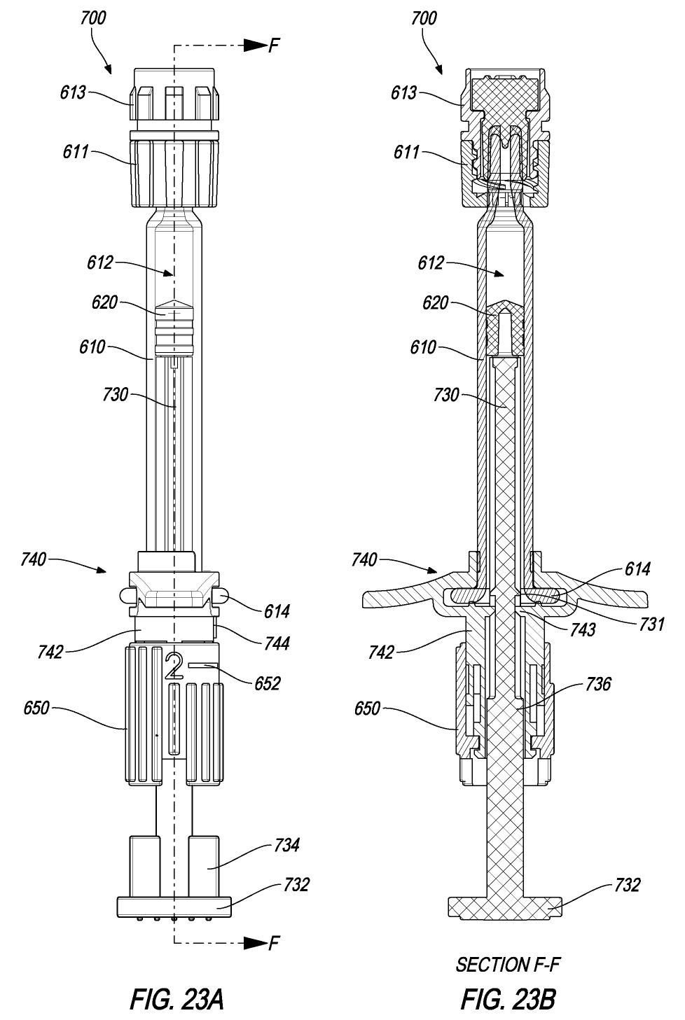

The plunger member 630 has a thumbpad 632 at a proximal end thereof and a distal end member 639 disposed adjacent to the stopper member 620. As shown in FIG. 7, the distal end member 639 has the form of a flange configured to interfere with the stopper member 620 and couple the plunger member 630 thereto. While the distal end member 639 of the plunger member 630 of the system depicted in FIGS. 6 and 7 has the form of a flange, in other embodiments the distal end of the plunger member may have a screw type connector to couple the plunger member to the stopper member. In still other embodiments (see FIGS. 22-23B described herein) the distal end of the plunger member may have a flat end configured to push the stopper member distally, but which may move proximally independent of the stopper member. The plunger member 630 also has proximal and distal pairs of externally extending ribs 634, 636 configured to cooperate with components of the dose setting collar 650 to control longitudinal movement of the plunger member 630 relative to the dose setting collar 650 and the finger flange 640.

The finger flange 640 defines a proximally extending member 642, which has a pointer 644 configured to cooperate with indicia 652 on the dose setting collar 650 to indicate various configurations of the system 600 (described below). As described above, the dose setting collar 650 has indicia 652 at a distal end thereof configured to cooperate with the pointer 644 on the proximally extending member 642 of the finger flange 640 indicate various configurations of the system 600 (described below). The indicia 652 may be printed, embossed, or engraved lines, graphics, text, numbers, icons, figures, and/or a combination thereof to indicate a configuration of the system 600. The dose setting collar 650 has a knurled external surface to facilitate manual rotation thereof.

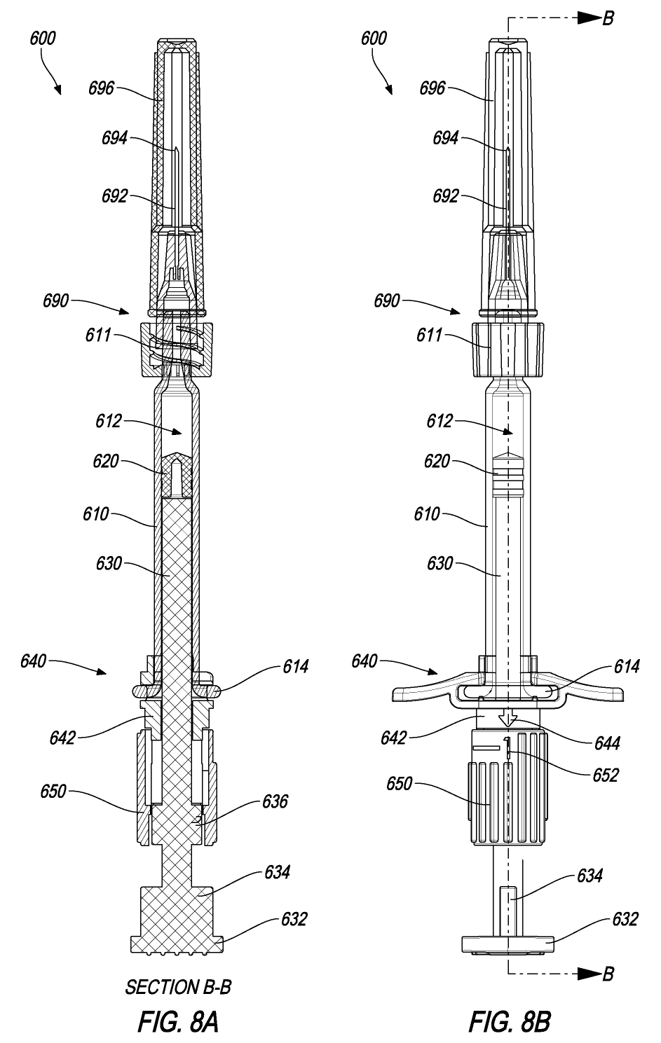

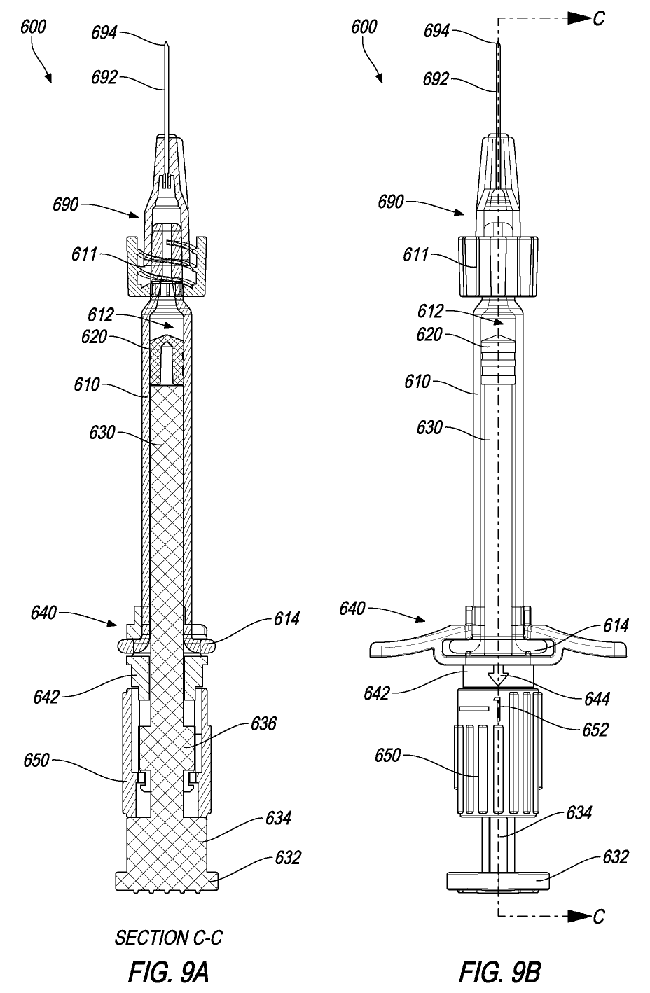

The system 600 may be configured to deliver a single 70 µl dose of medicine through the needle 692 (e.g., for intraocular injections). The syringe body 610 may be preloaded with approximately 200 µl (e.g., 170 µl to 230 µl) of medicine and an air bubble from filling (see e.g., FIGS. 8A and 8B). Purging of the needle 692 requires the expelling the air from the syringe body 610, and from the attached needle 692. Purging air from the system 600 leaves slightly more than 70 µl left in the syringe body 610 for dosing (see e.g., FIGS. 9A and 9B).

FIGS. 8A and 8B are longitudinal cross-sectional and side views depicting a system 600 for injecting in a transport configuration according to some embodiments. In the transport configuration, the system 600 may include a preinstalled needle hub assembly 690 as shown in FIGS. 8A and 8B. The needle hub assembly 690 may be removably coupled to the syringe body 610 using a needle interface 611 at the distal end of the syringe body 610. The needle interface 611 may be a female Luer connector and the needle hub assembly 690 may have a male Luer connector at a proximal end thereof. In some embodiments, in the transport configuration the system 600 may include a needle interface cover 613 coupled to the needle interface 611 and forming a fluid tight seal therewith as shown in FIGS. 6 and 7.

In the transport configuration, either a needle shield 696 sealing a sharp distal end 694 of a needle 692 in the needle hub assembly 690 or the needle interface cover 613 prevent liquids and air from escaping through the distal end of the syringe body 610 thereby limiting distal movement of the plunger member 630 and the stopper member 620 relative to the syringe body 610. In order to purge the system 600, the dose setting collar 650 is confirmed to be in state 1, or prime state, with the pointer 644 on the proximally extending member 642 of the finger flange 640 aligned with the number "1" in the indicia 652 on the dose setting collar 650, as shown in FIGS. 8A and 8B. The user may attach a needle hub assembly 690 to the needle interface 611 on the syringe body 610 if a needle hub assembly 690 is not already attached. The indicia may also be the word "Prime".

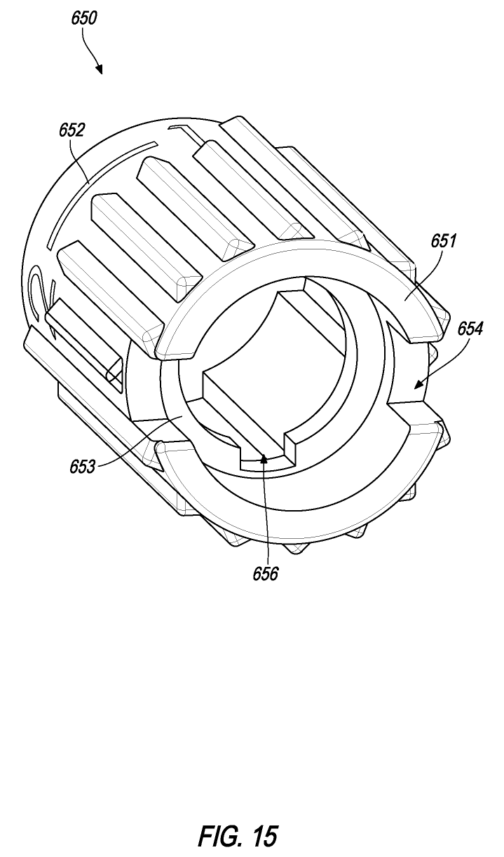

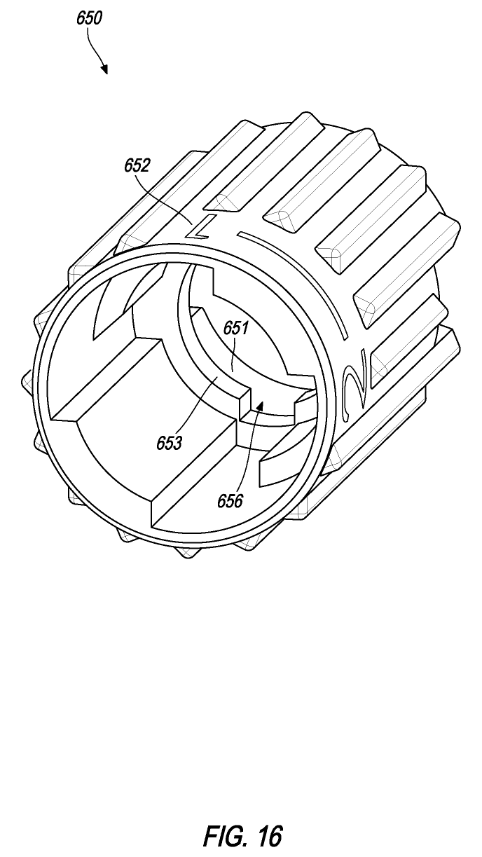

In the transport configuration, a distal pair of externally extending ribs 636 interferes with components of the dose setting collar 650 to prevent rotation of the dose setting collar 650 relative to the plunger member 630. In the transport configuration, the proximal pair of externally extending ribs 634 are aligned with a proximal pair of arcuate stops 651 in the dose setting collar 650 (see FIGS. 15 and 16) to limit distal advancement of the plunger member 630 and the stopper member 620 to the amount required to prime the system 600 by ejecting air from the syringe interior 612.

Priming the system 600 begins when the user positions the system 600 with the needle 692 pointing upward to allow any air to move to the distal end of the syringe interior 612 adjacent a proximal end of the needle 692. The user then advances the plunger member 630 distally relative to the finger flange 640 and the syringe body 610 and dose setting collar 650 coupled thereto (e.g., by pushing distally on the thumbpad 632) until the proximal externally extending ribs 634 under the thumbpad 632 abut a proximal end surface of the dose setting collar 650. As shown in FIGS. 9A and 9B, purging air from the system 600 leaves slightly more than 70 µl left in the syringe interior 612 for dosing. The system 600 in FIGS. 9A and 9B is in a primed configuration with substantially all of the air removed from the syringe interior 612.

In the primed configuration, distal movement of the plunger member 630 is prevented by interference between the pair of externally extending ribs 634 and the proximal pair of arcuate stops 651. In the primed configuration, the distal pair of externally extending ribs 636 has moved further distally into the dose setting collar 650, thereby allowing rotation of the dose setting collar 650 relative to the plunger member 630. The distal pair of externally extending ribs 636 performs a timing function by disabling and enabling rotation of the dose setting collar 650.

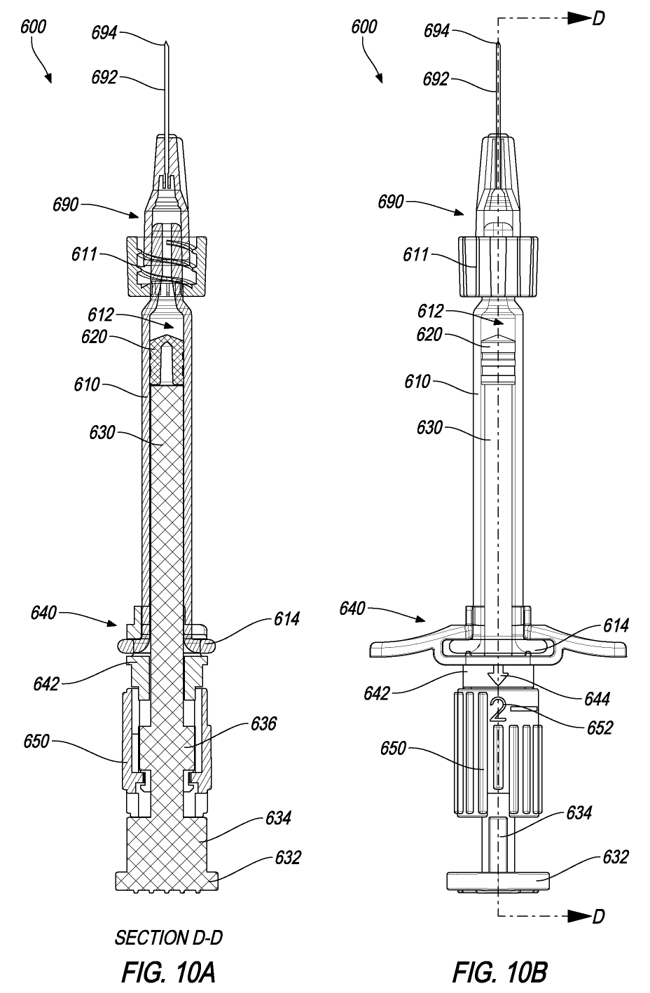

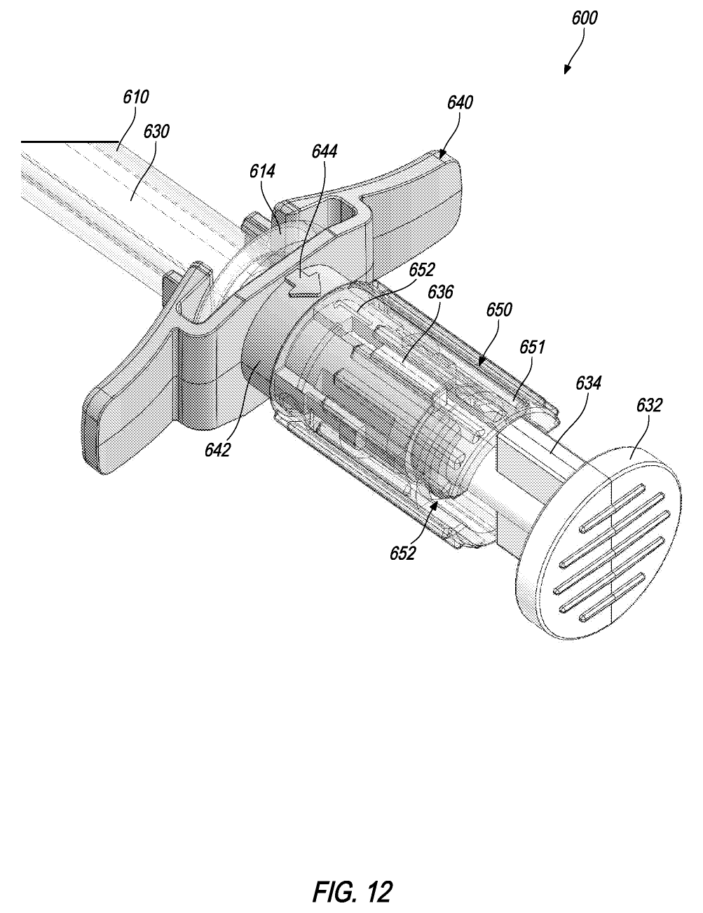

With the system 600 in the primed configuration, the user then rotates the dose setting collar 650 into state 2, or dose state, with the pointer 644 on the proximally extending member 642 of the finger flange 640 aligned with the number "2" in the indicia 652 on the dose setting collar 650, as shown in FIGS. 10A and 10B. Rotating the dose setting collar 650 into state 2 allows the proximal externally extending ribs 634, which was prevented from moving distally by proximal arcuate stop 651 (see FIG. 12), to move distally into a proximal longitudinal slot 654 in the dose setting collar 650 (see FIG. 12), thereby placing the system 600 in an injection ready configuration. The indicia may also be "Dose"

In the injection ready configuration, the proximal pair of externally extending ribs 634 are aligned with proximal pairs of longitudinal slots 654 defined by proximal pairs of arcuate stops 651 in an interior surface of the dose setting collar 650 (see FIGS. 15 and 16), which allows further distal advancement of the plunger member 630 and the stopper member 620 to deliver a dose of fluid/medicine through the needle 692.

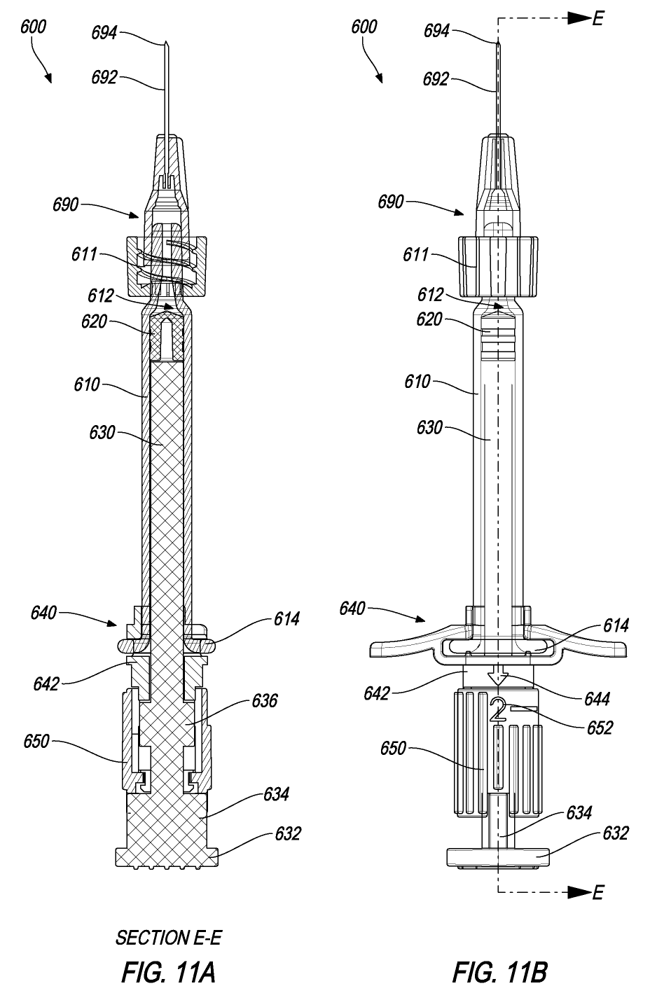

With the system 600 in the injection ready configuration, the user then further advances the plunger member 630 distally relative to the finger flange 640 and the syringe body 610 and dose setting collar 650 coupled thereto (e.g., by further pushing distally on the thumbpad 632) until the proximal externally extending ribs 634 under the thumbpad 632 abut a proximally facing surface at respective distal ends of the proximal pair of longitudinal slots 654. Further advancing the plunger member 630 distally delivers the dose of fluid/medicine through the needle 692 thereby placing the system 600 in an injected configuration as shown in FIGS. 11A and 11B . The system 600 is configured to eject a portion of the fluid/medicine that is less than a total amount of the fluid/medicine in the syringe interior 612 to accommodate tolerances in various components of the system 600.

The plunger member 630 and the dose setting collar 650 have various features, including proximal and distal pairs of externally extending ribs 634, 636 and proximal and distal pairs of longitudinal slots 654, 656, respectively along their lengths to provide various controls/limits to translational and rotational movements of the dose setting collar 650 relative to the plunger member 630 in various configurations of the system 600. In particular, the system 600 is configured such that the dose setting collar 650 is prevented from rotating until air has been purged from the syringe interior 612 to prevent inadvertent dosing.

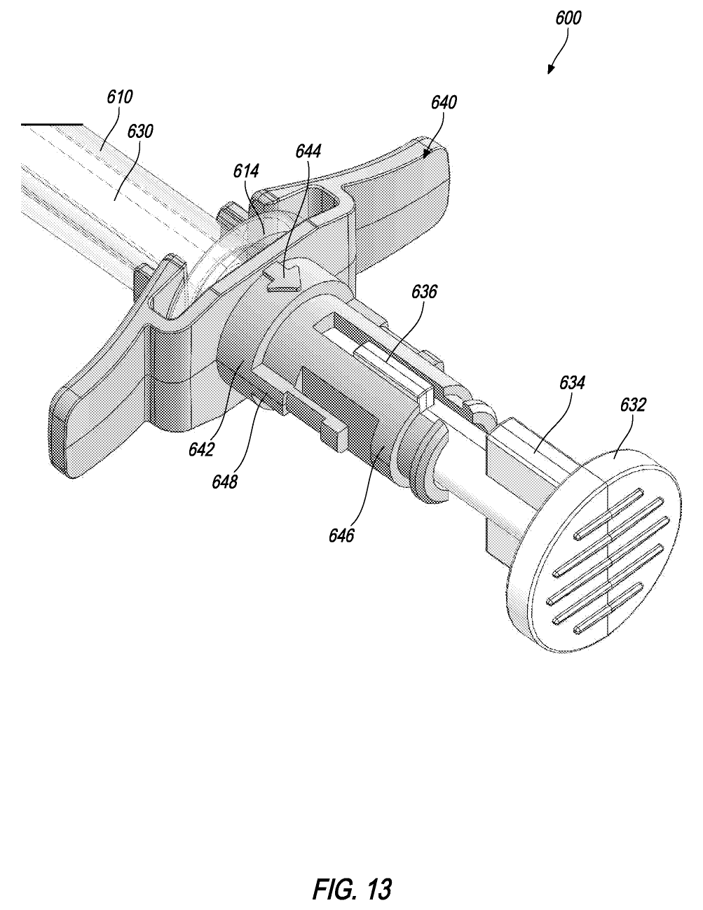

FIG. 12 is a detailed perspective view depicting a system 600 for injecting in a primed configuration according to some embodiments, with the dose setting collar 650 shown in shadow. In the primed configuration, distal movement of the plunger member 630 is prevented by interference between the pair of externally extending ribs 634 and the proximal pair of arcuate stops 651. FIG. 13 is a detailed perspective view depicting a system 600 for injecting in a primed configuration according to some embodiments, with the dose setting collar (650 see FIG. 12) omitted for clarity. The proximally extending member 642 of the finger flange 640 includes a pair of flex arms 646 to provide positional detents inside the dose setting collar 650. The flex arms 646 also provide audible and/or tactile cues/feedback corresponding to rotation of the dose setting collar 650. The proximally extending member 642 also defines a pair of rotational stop ribs 648 to provide a hard stop when rotating the dose setting collar 650 to move from state 1/"Prime" configuration to state 2/injection ready "Dose" configuration.

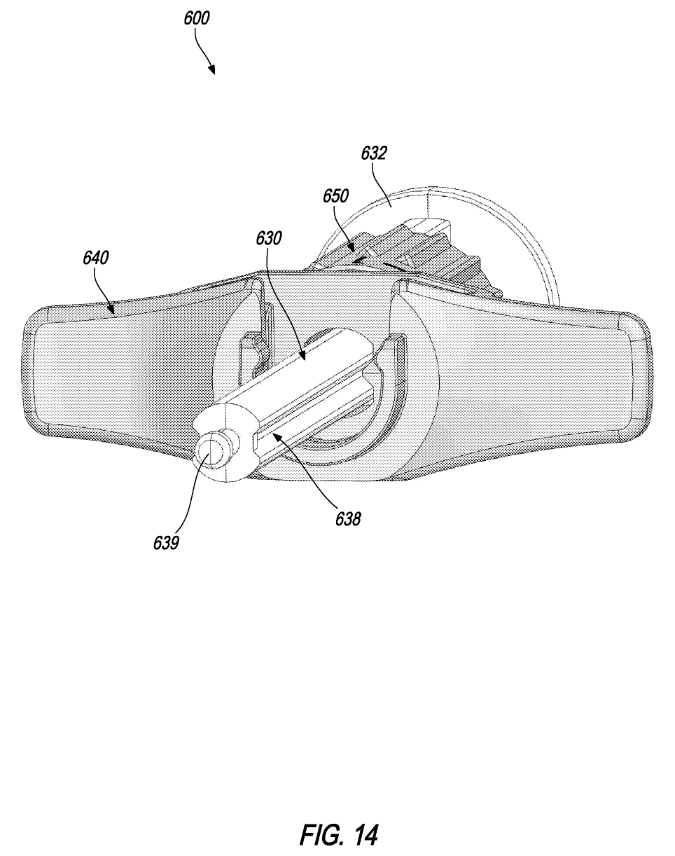

FIG. 14 is a detailed perspective view depicting a system 600 for injecting in a primed configuration according to some embodiments, with the syringe body and stopper member (610 and 620 see FIG. 6) omitted for clarity. The plunger member 630 defines a keyed channel 638 configured to cooperate with a plunger rotation stop 641 defined by the finger flange 642 prevent rotation of the plunger member 630 relative to the finger flange 642. Because the plunger member 630 cannot rotate relative to the finger flange 642, the distal end member 639 of the plunger member has the form of a flange configured to be inserted into and interfere with the stopper member 620 to couple the plunger member 630 thereto.

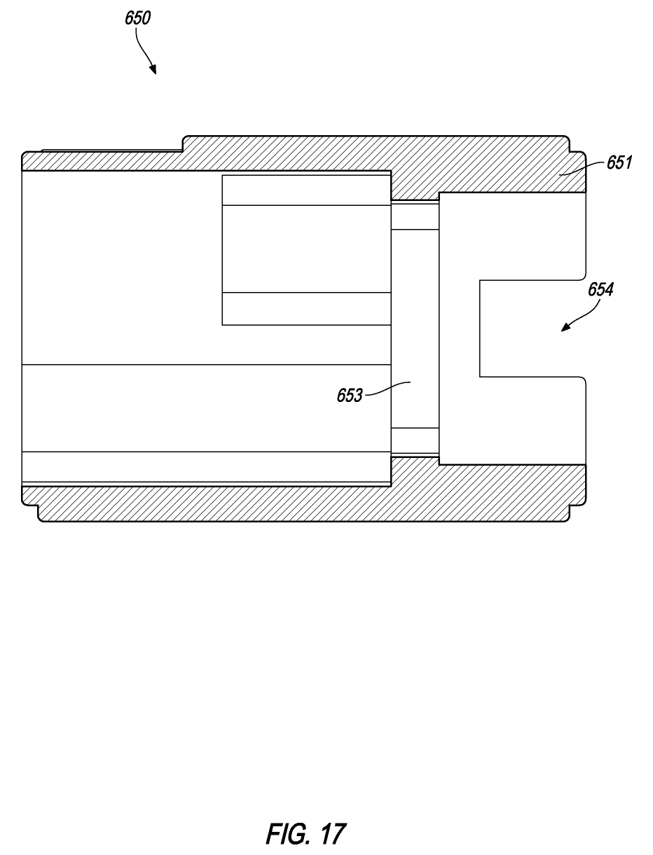

FIGS. 15 and 16 are proximal and distal perspective views depicting a dose setting collar 650 for use with systems for injecting according to some embodiments. FIG. 17 is a longitudinal cross-sectional view depicting a dose setting collar 650 for use with systems for injecting according to some embodiments. FIGS. 15 to 17 show that proximal and distal pairs of longitudinal slots 654, 656 are defined by respective proximal and distal pairs of arcuate stops 651, 653 in an interior surface of the dose setting collar 650.

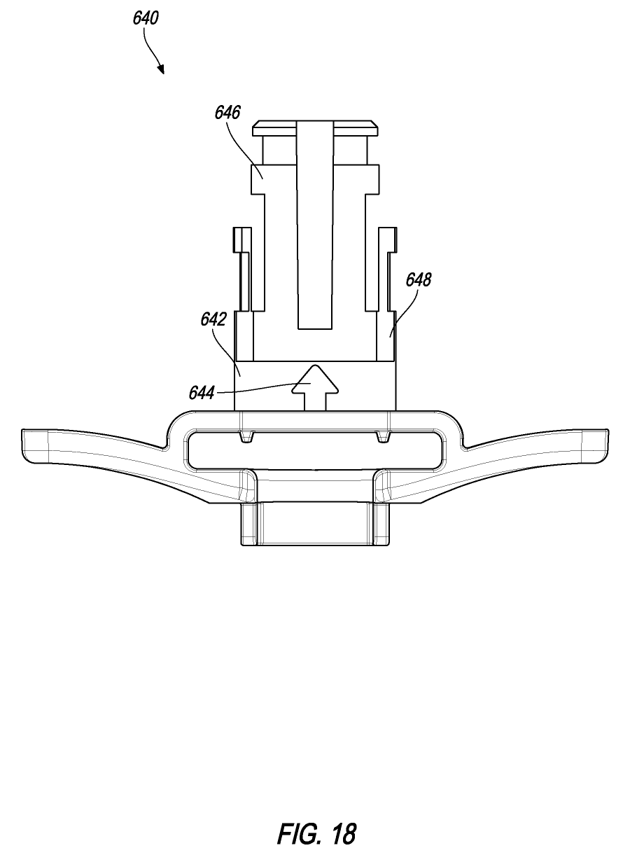

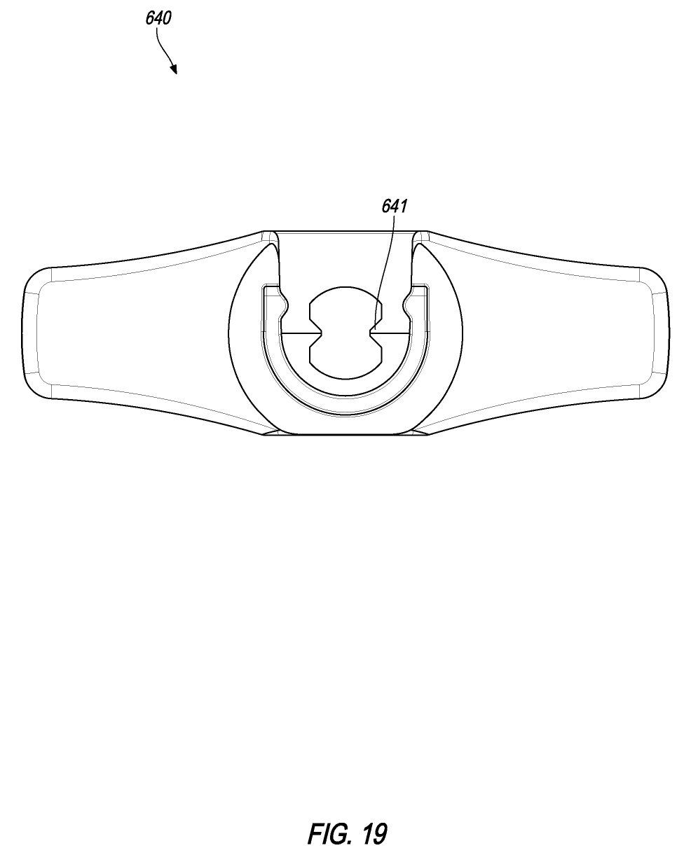

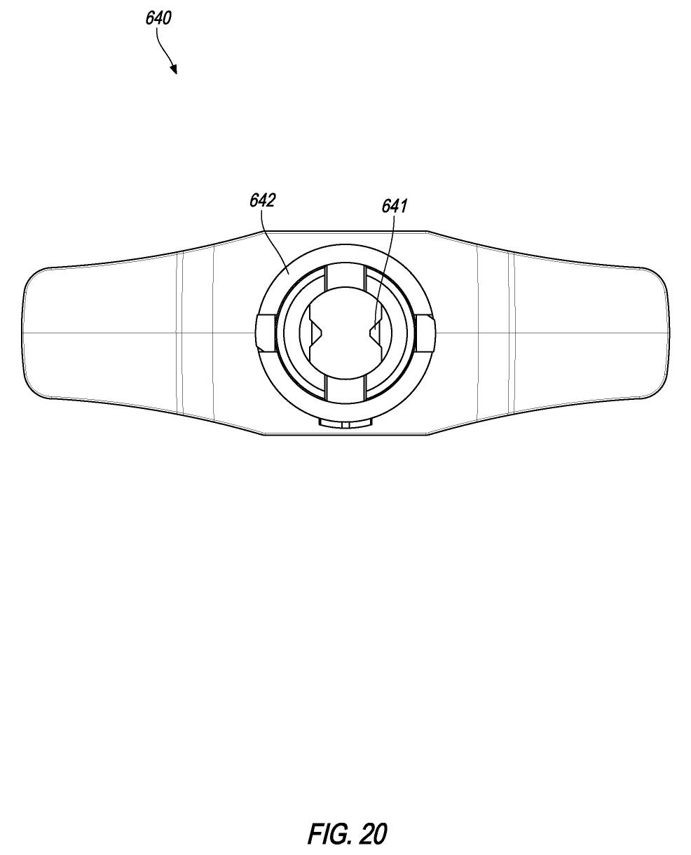

FIGS. 18, 19, and 20, are side, bottom, and top views depicting a finger flange for use with systems for injecting according to some embodiments. The proximally extending member 642 of the finger flange 640 includes a pair of flex arms 646 to provide positional detents inside the dose setting collar 650. The flex arms 646 also provide audible and/or tactile cues/feedback corresponding to rotation of the dose setting collar 650. The proximally extending member 642 also defines a pair of rotational stop ribs 648 to provide a hard stop when rotating the dose setting collar 650 to move from state 1/"Prime" configuration to state 2 / injection ready "Dose" configuration.

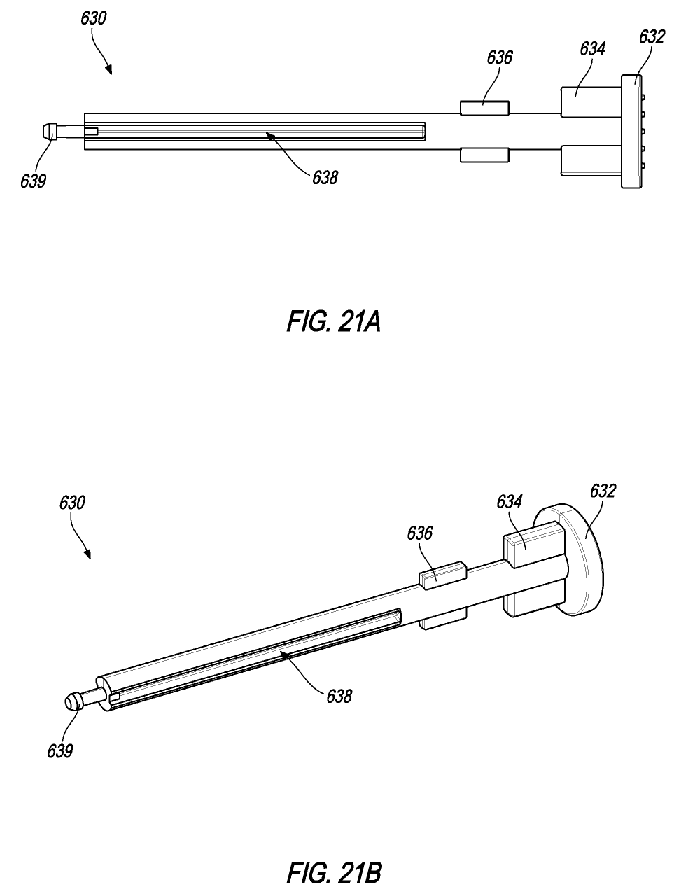

FIGS. 21A and 21B are side and perspective views depicting a plunger member 630 for use with systems for injecting according to some embodiments. The plunger member 630 includes a thumbpad 632, proximal and distal externally extending ribs 634, 636, a keyed channel 638, and a distal end member 639.

FIG. 22 is a perspective view depicting a plunger member 730 for use with systems for injecting according to some embodiments. The plunger member 730 includes a thumbpad 732, proximal and distal externally extending ribs 734, 736, a keyed channel 738, and a flat distal end 739. The difference between the plunger members 730 depicted in FIG. 22 and the plunger member 630 depicted in FIGS. 21A and 21B is that plunger member 730 has a flat distal end 739 whereas plunger member 630 has a distal end member 639 with the form of a flange configured to couple the plunger member 630 to the stopper member 620. As such, plunger member 730 is configured to push stopper member 620 distally while being free to move proximally independent of stopper member 620.

FIGS. 23A and 23B are side and longitudinal cross-sectional views depicting a system 700 for injecting in an injection ready configuration according to some embodiments. The system 700 depicted in FIGS. 23A and 23B is very similar to the system 600 depicted in FIGS. 6 to 21B except that the plunger member 730 has a flat distal end 739. Components of system 700 have similar numbers to corresponding components of system 600.

The system 700 includes a syringe body 610 having proximal and distal ends, a syringe interior 612, and a syringe flange 614 at the proximal end thereof. A stopper member 620 is disposed in the syringe interior 612 to form a fluid chamber therein. The syringe interior/fluid chamber 612 contains an injectable fluid (not shown) and some air. A plunger member 730 is partially disposed in the syringe interior 612. The plunger member 730 includes a thumbpad 732, proximal and distal externally extending ribs 734, 736, a keyed channel 738, and a flat distal end 739. The system 700 also includes a finger flange 740 coupled to the syringe flange 612. The system 700 further includes a dose setting collar 650 coupled to the finger flange 640 and the plunger member 730.

The syringe body 610, stopper member 620, and dose setting collar 650 are identical to the syringe body 610, stopper member 620, and dose setting collar 650 of the system 600 described herein. The plunger member 730 and finger flange 640 are very similar to the plunger member 630 and shroud 640 of the system 600 described herein. One difference is that the plunger member 730 in system 700 has a flat distal end 739 instead of a distal end member 639 with the form of a flange configured to couple the plunger member 630 to the stopper member 620 as in system 600.

Because the plunger rod 730 is free to move proximally independent of stopper member 620, the plunger rod 730 may unintentionally disengage from system 700 during transport. As such, plunger rod includes at least one and/or a plurality of barbs 731 configured to interfere with ribs on the finger flange to prevent unintentional disengagement.

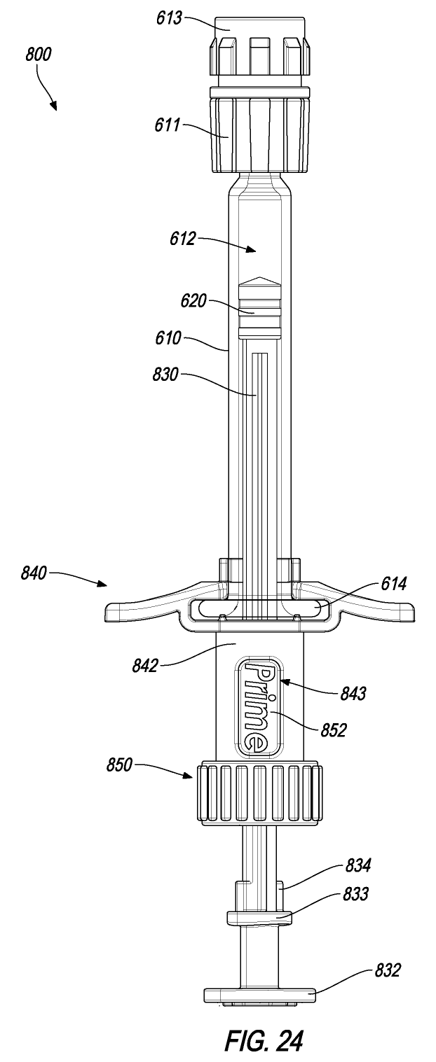

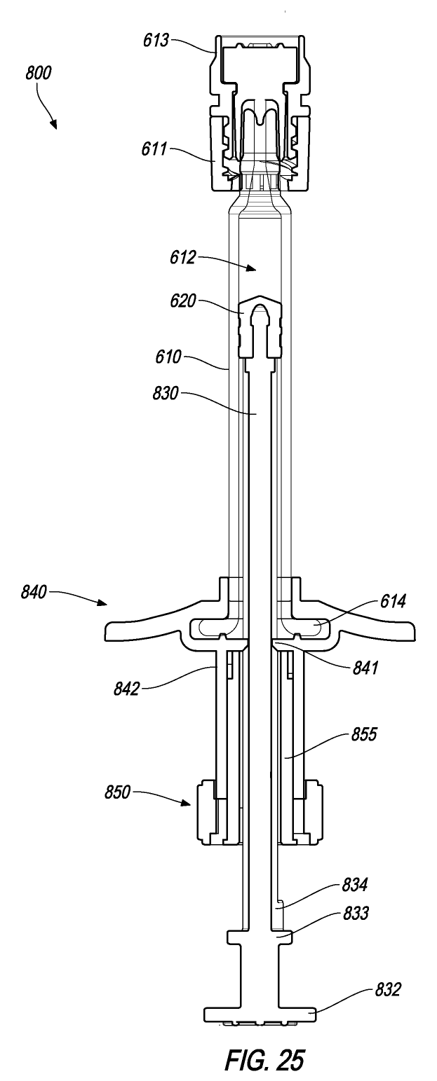

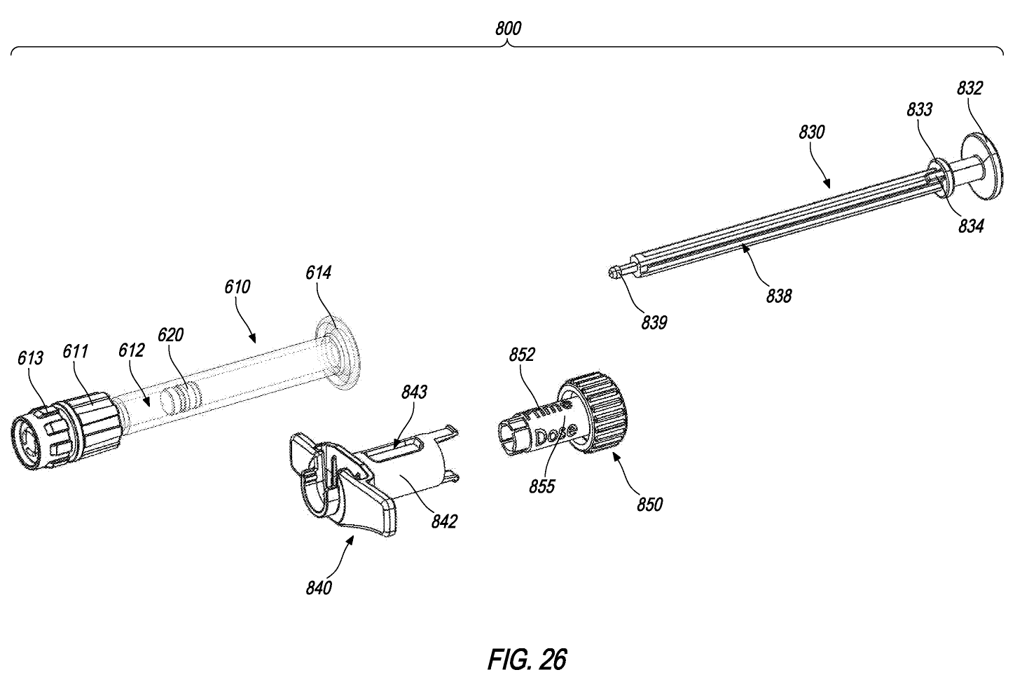

FIGS. 24, 25, and 26 are side, longitudinal cross-sectional, and exploded views depicting a system 800 for injecting according to some embodiments. The system 800 depicted in FIGS. 24, 25, and 26 is very similar to the systems 600, 700 depicted in FIGS. 6 to 21B and 22 to 23B. Components of system 800 have similar numbers to corresponding components of system 600. The main difference is that the system 800 has a dose setting collar 850 with a distally extending member 855 configured to extend into a proximally extending member 842 of the finger flange 840 (see e.g., FIG. 26).

The proximally extending member 842 of the finger flange 840 of the system 800 also defines at least one indicia viewing window 843 extending through the proximally extending member 842. The indicia viewing window 843 is configured to align with indicia 852 on the distally extending member 855 of the dose setting collar 850 to display the current mode of the dose setting collar 850.

The system 800 includes a syringe body 610 having proximal and distal ends, a syringe interior 612, and a syringe flange 614 at the proximal end thereof. A stopper member 620 is disposed in the syringe interior 612 to form a fluid chamber therein. The syringe interior/fluid chamber 612 contains an injectable fluid (not shown) and some air. A plunger member 830 is partially disposed in the syringe interior 612.

The syringe body 610 and stopper member 620 are identical to the syringe body 610 and stopper member 620 of the system 600 described herein. The plunger member 830, finger flange 840, and dose setting collar 850 are very similar to the plunger member 630, finger flange 640, and does setting collar 640 of the system 600 described herein.

As shown in FIG. 26, the plunger member 830 includes a thumbpad 832, a dose flange 833, a dose setting rib 834, a keyed channel 838, and a distal end member 839. In an alternative embodiment (not shown), the plunger member, may be configured with a flat distal end for engagement with the stopper member and at least one and/or a plurality of barbs (see e.g., FIG. 22) configured to interfere with ribs on the finger flange to prevent unintentional disengagement. The system 800 also includes a finger flange 840 coupled to the syringe flange 612. The system 800 further includes a finger flange coupled to the syringe flange 614 of the syringe body 610 and a dose setting collar 850 coupled to the finger flange 840 and the plunger member 830.

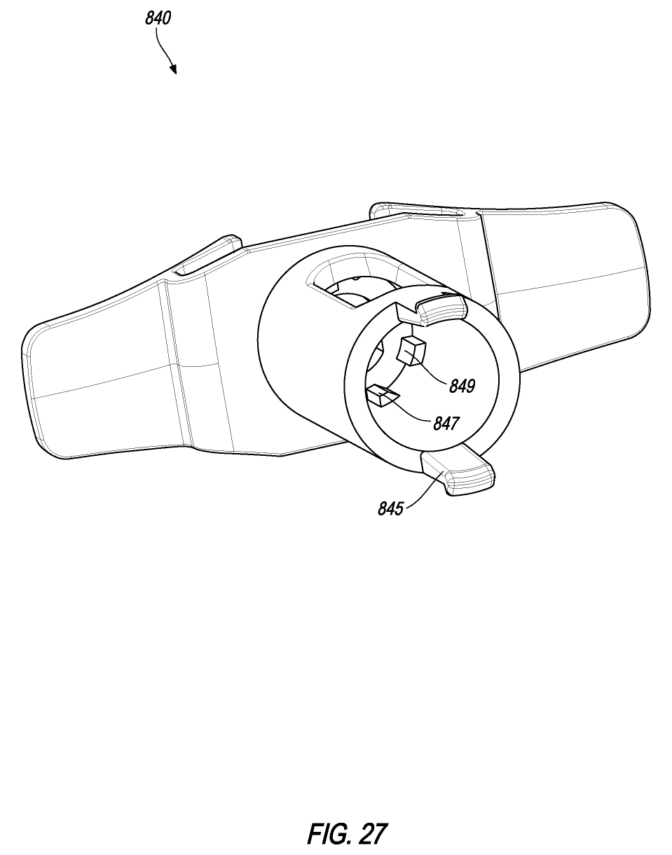

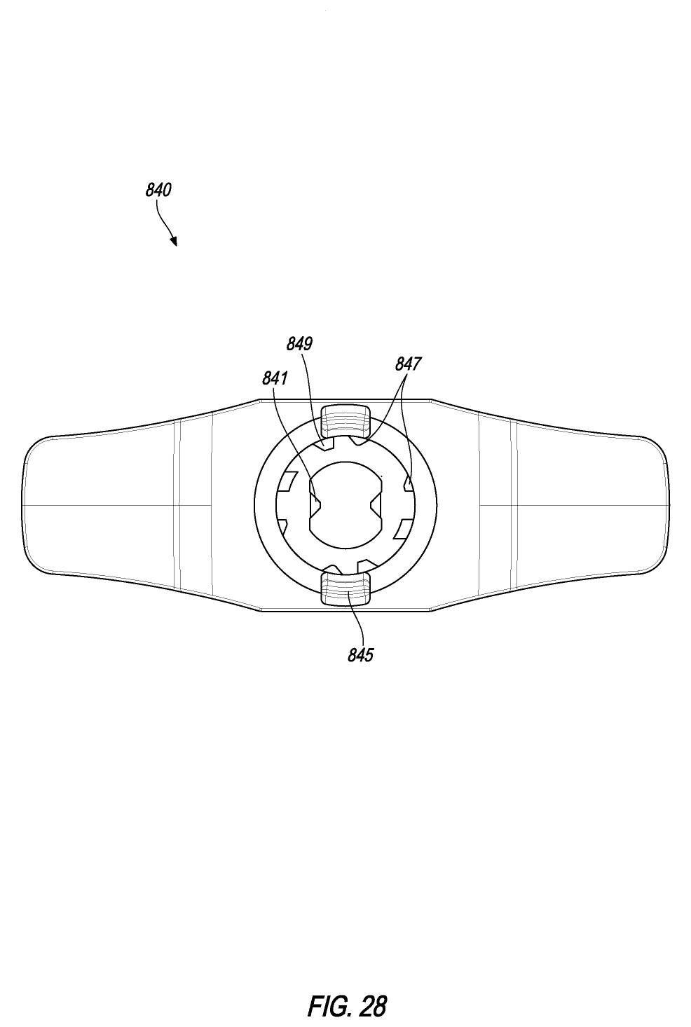

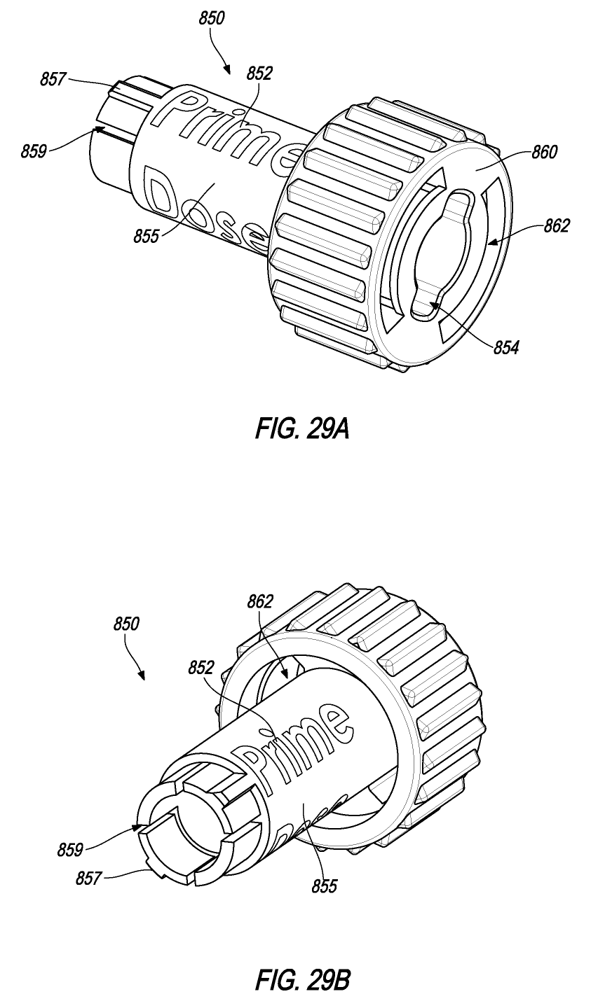

FIGS. 27 and 28 are perspective and top views depicting a finger flange 840 for use with systems 800 for injecting according to some embodiments. The finger flange 840 includes a proximally extending member 842 and defines detent ribs 847 and rotational stop ribs 849 on an interior surface of the proximally extending member 842. The detent ribs 847 are configured to interfere with axially extending ribs 857 on the dose setting collar item 850 (see e.g., FIGS. 29A and 29B) to provide audible and/or tactile feedback (e.g., a click) to the user when switching modes. The rotational stop ribs 849 are configured to stop rotation of the dose setting collar 850 at a predetermined degree of rotation where the indicia are aligned with and visible through the indicia viewing window 843. The finger flange 840 also includes assembly snaps 845, configured to couple the dose setting collar 850 to the finger flange 840. The assembly snaps are configured to elastically deflect radially inward and snap outward into circumferential groves 862 in the dose setting collar 850, allowing the dose setting collar 850 to rotate about a longitudinal axis while preventing axial movement relative to the finger flange 840.

FIGS. 29A and 29B are proximal and distal perspective views depicting a dose setting collar 850 for use with systems 800 for injecting according to some embodiments. The dose setting collar 850 defines the axially extending ribs 857 and at least one axially extending slots 859 therebetween, which allow the axially extending ribs 857 to flex radially inward when overcoming the interference of the detent ribs 847 during rotation of the dose setting collar 850 relative to the finger flange 840. Indicia 852 are printed, embossed, or etched on the outer surface of the distally extending member 855, which are configured to align with the indicia viewing window 843 to display an indication to the user of the state of the system 800. The indicia 852 may be text as shown, or may be graphics, numbers, or alpha-numeric indicators. While two indicia are shown, one, three, or more indicia may be used in some embodiments.

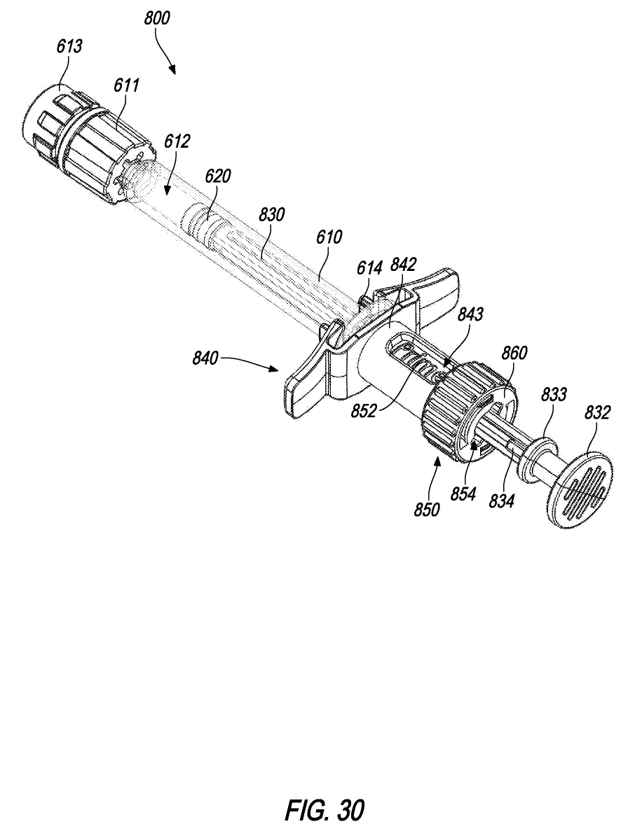

FIG. 30 is a perspective view depicting a system 800 for injecting in a transport configuration according to some embodiments. In the transport configuration, the system 800 may include may include a needle interface cover 613 coupled to the needle interface 611 and forming a fluid tight seal therewith. The needle interface 611 may be a female Luer connector and the needle hub assembly 690 may have a male Luer connector at a proximal end thereof. In the transport configuration, the needle interface cover 613 prevents liquids and air from escaping through the distal end of the syringe body 610 thereby limiting distal movement of the plunger member 830 and the stopper member 620 relative to the syringe body 610. In order to purge the system 800, the dose setting collar 850 is confirmed to be in a "Prime" state with the "Prime" indicia 852 on the distally extending member 855 of the dose setting collar 850 aligned with the indicia viewing window 843 on the proximally extending member 842 of the finger flange 840, as shown in FIG. 30.

In the transport configuration, the dose setting rib 834 is out of alignment with a proximal longitudinal slot 854 in the dose setting collar 850 to limit distal advancement of the plunger member 830 and the stopper member 620 to the amount required to prime the system 800 by ejecting air from the syringe interior 612. Similar to the embodiments in FIGS. 6 to 13 the plunger member 830 define distal externally extending ribs (not shown, see 636 in FIG. 7), which cooperate with internal ribs (not shown) in the dose setting collar 850 to limit rotation of the dose setting collar 850 until the plunger member 830 is moved distally into the primed configuration.

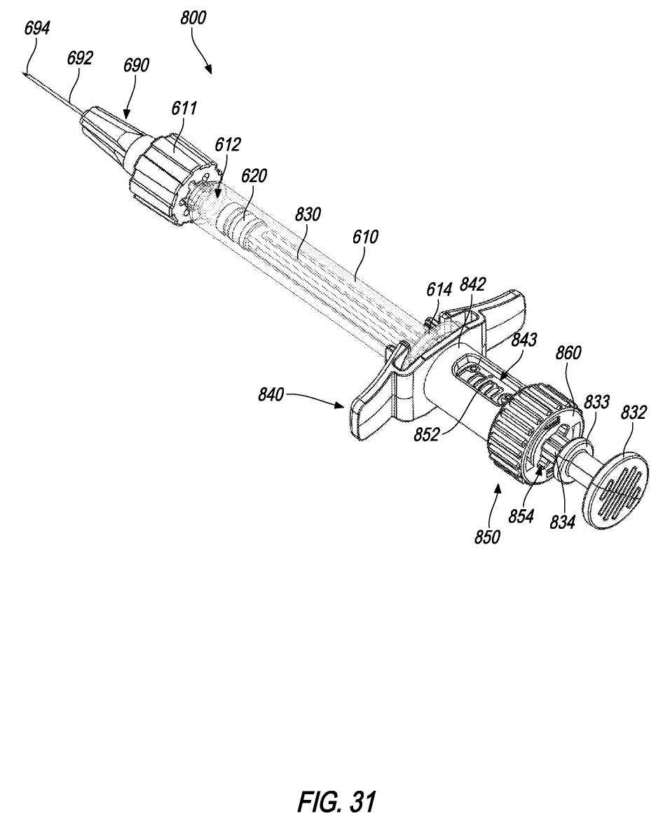

Priming the system 800 begins when the user attaches a needle hub assembly 690 to the needle interface 611 on the syringe body 610, and positions the system 800 with the needle 692 pointing upward to allow any air to move to the distal end of the syringe interior 612 adjacent a proximal end of the needle 692, as shown in FIG. 31. The user then advances the plunger member 830 distally relative to the finger flange 840 and the syringe body 610 and dose setting collar 850 coupled thereto (e.g., by pushing distally on the thumbpad 832) until a distal end of the dose setting rib 834 (under dose flange 833 distally spaced from the thumbpad 832) abuts a proximal end surface 860 of the dose setting collar 850. As shown in FIG. 31, purging air from the system 800 leaves slightly more than 70 µl left in the syringe interior 612 for dosing. The system 800 in FIG. 31 is in a primed configuration with substantially all of the air removed from the syringe interior 612. While 70 µl is the target dose for the devices depicted here, smaller or larger target doses between approximately 5 µl and 400 µl may be used in some embodiments. Similar to the embodiments in FIGS. 6 to 13 the plunger member 830 define distal externally extending ribs (not shown, see 636 in FIG. 7), which cooperate with internal ribs (not shown) in the dose setting collar 850 to limit rotation of the dose setting collar 850 until the plunger member 830 is moved distally into the primed configuration.

In the primed configuration, distal movement of the plunger member 830 is prevented by interference between the dose setting rib 834 and the proximal end surface 860 of the dose setting collar 850. In the primed configuration, rotation of the dose setting collar 850 relative to the plunger member 830 is allowed.

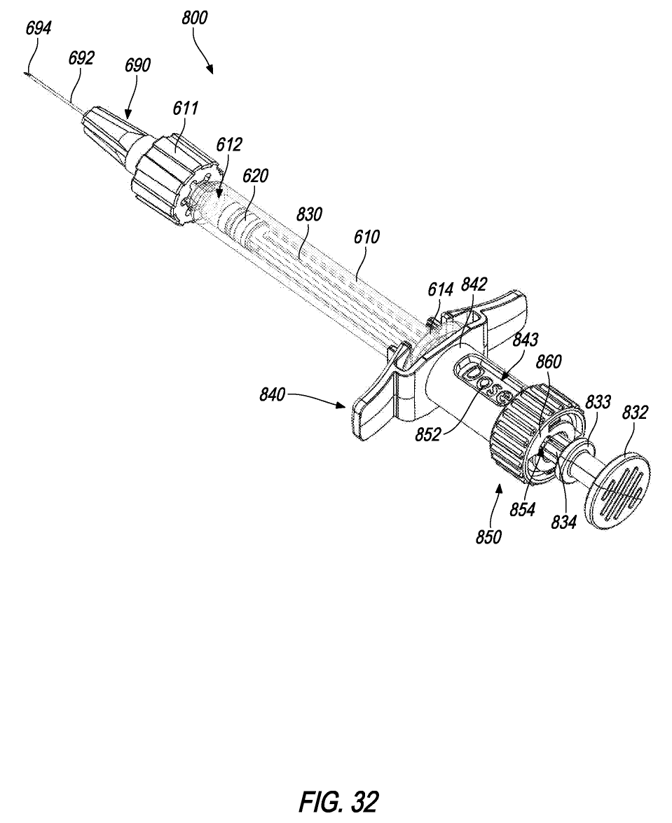

With the system 800 in the primed configuration, the user then rotates the dose setting collar 850 into a "Dose" state with the "Dose" indicia 852 on the distally extending member 855 of the dose setting collar 850 aligned with the indicia viewing window 843 on the proximally extending member 842 of the finger flange 840, as shown in FIG. 32.. Rotating the dose setting collar 850 into the "Dose" state allows the dose setting rib 834, which was prevented from moving distally by the proximal end surface 860 of the dose setting collar 850 (see FIG. 30), to move distally into a proximal longitudinal slot 854 in the dose setting collar 850 (see FIG. 31), thereby placing the system 800 in an injection ready configuration. The rotation of the dose setting collar 850 into the "Dose" state also causes an interference between the detent ribs 847 and the axially extending ribs 857 causing radial deformation of the axially extending ribs 857 from a rest state to a deformed state. Upon further rotation of the dose setting collar 850, the axially extending ribs 857 pass the detent ribs 847 and snap back to their initial state, causing one or more audible and/or tactile clicks to provide additional user feedback as to the state of the dose setting collar 850.

In the injection ready configuration, the dose setting rib 834 is aligned with the proximal longitudinal slot 854 defined by the dose setting collar 850 (see FIGS. 29A and 29B), which allows further distal advancement of the plunger member 830 and the stopper member 620 to deliver a dose of fluid/medicine through the needle 692.

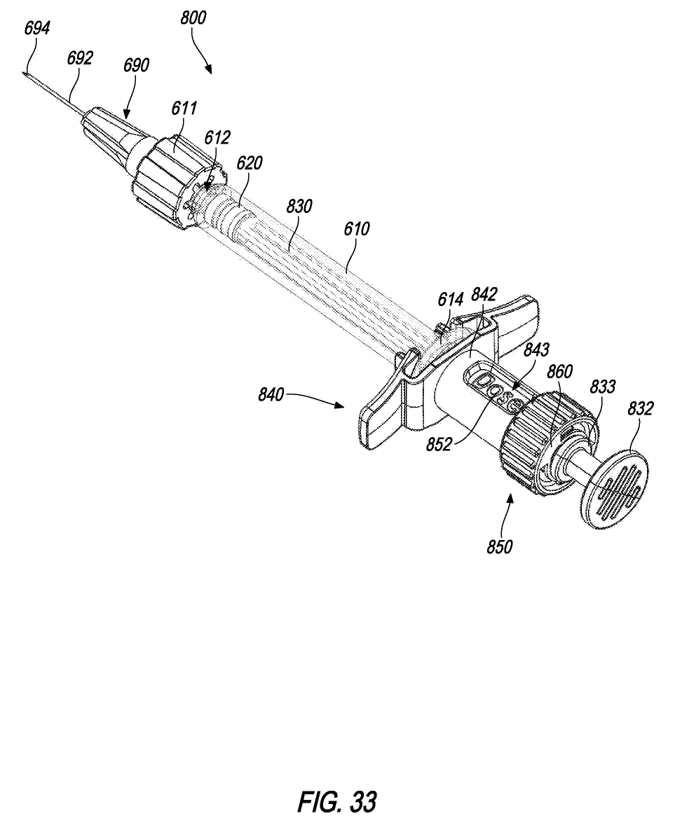

With the system 800 in the injection ready configuration, the user then further advances the plunger member 830 distally relative to the finger flange 840 and the syringe body 610 and dose setting collar 850 coupled thereto (e.g., by further pushing distally on the thumbpad 832) until the dose flange 833 distally spaced from the thumbpad 832 abuts the proximal end surface 860 of the dose setting collar 850. Further advancing the plunger member 830 distally delivers the dose of fluid/medicine through the needle 692 thereby placing the system 800 in an injected configuration as shown in FIG. 33. The system 800 is configured to eject a portion of the fluid/medicine that is less than a total amount of the fluid/medicine in the syringe interior 612 to accommodate tolerances in various components of the system 800.

Like many of the injection systems described in co-owned U.S. patent application serial nos. 14/696,342, 14/543,787, 14/321,706, 62/416,102, 62/431,382, 62/480,276, 62/508,508, 62/542,230, 15/801,239, 15/801,259, 15/801,281, 15/801,304, 16/011,453, 16/683,126, and 16/683,157, the contents of which were previously fully incorporated herein by reference as though set forth in full, the systems 800, 700 utilizes off-the-shelf syringe bodies 610, 710, stopper members 620, 720, and needle connection members. The injection systems 800, 700 may also be used with off-the-shelf needle assemblies including needle hubs and needles.

While various embodiments have been described with specific connectors (e.g., slip and Luer), these embodiments can be used with any known injection system connectors. While various embodiments have been described with staked needles and needle connectors, these embodiments can be used with any known permanently coupled needle or needle connector system.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply "kits" may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the "providing" act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above- referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms "a," "an," "said," and "the" include plural referents unless the specifically stated otherwise. In other words, use of the articles allow for "at least one" of the subject item in the description above as well as claims associated with this disclosure. It is further noted that such claims may be drafted to exclude any optional element. As such, this statement is intended to serve as antecedent basis for use of such exclusive terminology as "solely," "only" and the like in connection with the recitation of claim elements, or use of a "negative" limitation.

Without the use of such exclusive terminology, the term "comprising" in claims associated with this disclosure shall allow for the inclusion of any additional element--irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for injecting, comprising:
a syringe body having proximal and distal ends, a syringe interior, and a syringe flange at the proximal end thereof;
an injectable fluid disposed in the syringe interior;
a stopper member disposed in the syringe interior;
a plunger member having
a thumbpad at a proximal end thereof, and
a distal end configured to be disposed adjacent to the stopper member;
a finger flange removably coupled to the syringe flange; and
a collar rotatably coupled to the finger flange and disposed around the plunger member to control movement of the plunger member relative to the finger flange.

2. The system of claim 1, wherein the plunger member is keyed to the finger flange to prevent rotation of the plunger member relative to the finger flange.

3. The system of claim 1, wherein the distal end of the plunger member is coupled to the stopper member.

4. The system of claim 1, wherein the finger flange comprises:
a proximally extending member having a pair of flex arms configured to provide an audible and/or tactile cue during rotation of the collar about the proximally extending member; and
a rotational stop rib configured to limit rotation of the collar about the proximally extending member.

5. The system of claim 4, wherein the collar comprises:
a pair of proximally facing proximal arcuate stops that define a corresponding pair of proximal longitudinal slots; and
a pair of proximally facing distal arcuate stops that define a corresponding pair of distal longitudinal slots, and
wherein the proximally facing proximal arcuate stops and the proximally facing distal arcuate stops are rotationally displaced from each other.

6. The system of claim 5, wherein the plunger member comprises proximal and distal pairs of externally extending ribs configured to cooperate with the proximally facing proximal and distal arcuate stops to control longitudinal movements of the plunger member relative to the finger flange.

7. The system of claim 6, wherein the distal pair of externally extending ribs are configured to control rotation of the collar.

8. The system of claim 6, wherein the proximal and distal pairs of externally extending ribs define a respective pair of longitudinal spaces therebetween.

9. The system of claim 1, wherein the collar comprises a proximally facing stop surface defining a central opening.

10. The system of claim 9, wherein the plunger member comprises a pair of externally extending ribs configured to cooperate with the proximally facing stop surface and the central opening to control longitudinal movements of the plunger member relative to the finger flange.

11. The system of claim 1, wherein the system has:
a transport configuration in which the plunger member is in a proximal most position,
a primed/injection ready configuration in which the plunger member is in an intermediate position,
an injected configuration in which the plunger member is in a distal most position,
a first stage in which the system can convert from the transport configuration to the primed/injection ready configuration, and
a second stage in which the system can convert from the primed/injection ready configuration to the injected configuration.

12. The system of claim 11, wherein the finger flange defines a pointer,
wherein the collar defines a first external indicia of the first stage and a second external indicia of the second stage, and
wherein the pointer on the finger flange is configured to align with the first external indicia on the collar when the system is in the first stage and align with the second external indicia on the collar when the system is in the second stage.

13. The system of claim 11, wherein the system is configured to transition from the first stage to the second stage by rotation of the collar relative to the finger flange.

14. The system of claim 11, wherein in the injected configuration, less than a total amount of the injectable fluid is ejected from the syringe interior.

15. The system of claim 1, wherein the collar defines an external indicia of rotation direction.

16. The system of claim 1, wherein the plunger rod comprises a barb configured to interact with a rib on the finger flange to limit proximal movement of the plunger rod relative to the finger flange.

17. The system of claim 1, wherein the collar is disposed longitudinally between the thumbpad and the finger flange.

18. The system of claim 1, wherein the collar comprises a distally extending member having an external indicia of system mode defined thereon,
wherein the finger flange comprises a proximally extending member defining a viewing window configured to align with and display the indicia of system mode defined on the distally extending member of the collar, and
wherein the distally extending member of the collar is configured to be at least partially disposed coaxially within the proximally extending member of the finger flange.

19. The system of claim 18, wherein the indicia of system mode and the viewing window are configured to indicate a prime mode and a dose mode of the system.

20. The system of claim 18, wherein the distally extending member of the collar defines an axially extending rib, and
wherein the proximally extend member of the finger flange defines a detent rib and a rotational stop rib.

21. The system of claim 20, wherein the axially extending rib is configured to cooperate with the detent rib to generate an audible/tactile click during a mode switch.

22. The system of claim 20, wherein the axially extending rib is configured to cooperate with the rotational stop rib to limit rotation of the collar relative to the finger flange.

23. A method for injecting a fluid, comprising:
providing a syringe assembly, the syringe assembly comprising
a syringe body having proximal and distal ends, a syringe interior, and a syringe flange at the proximal end thereof;
an injectable fluid disposed in the syringe interior;
a stopper member disposed in the syringe interior;
a plunger member having
a thumbpad at a proximal end thereof, and
a distal end configured to be disposed adjacent to the stopper member;
a finger flange removably coupled to the syringe flange; and
a collar rotatably coupled to the finger flange and disposed around the plunger member to control movement of the plunger member relative to the finger flange,
wherein the syringe assembly has a first stage configured for transport and purging of air in the syringe interior, and
wherein the syringe assembly has a second stage configured for delivery of a portion of the injectable fluid in the syringe interior;
with the syringe assembly in the first stage, applying a first distally directed force to the thumbpad to advance the plunger member and the stopper member to a first distal end position to expel air from the syringe interior through the distal end of the syringe body;
rotating the collar relative to the finger flange to a rotation end location to transform the syringe assembly from the first stage to the second stage; and
with the syringe assembly in the first stage, applying a second distally directed force to the thumbpad to advance the plunger member and the stopper member to a second distal end position to expel the portion of the injectable fluid from the syringe interior through the distal end of the syringe body.

24. The method of claim 23, wherein the syringe assembly has:
a transport configuration in which the plunger member is in a proximal most position,
a primed/injection ready configuration in which the plunger member is in the first distal end position, and
an injected configuration in which the plunger member is in the second distal end position,
wherein applying the first distally directed force to the thumbpad to advance the plunger member and the stopper member to the first distal end position and rotating the collar relative to the finger flange to the rotation end location transforms the syringe assembly from the transport configuration to the primed/injection ready configuration, and
wherein applying the first distally directed force to the thumbpad to advance the plunger member and the stopper member to the first distal end position transforms the syringe assembly from the primed/injection ready configuration to the injected configuration.

25. The method of claim 24, wherein the collar defines a first external indicia of the first stage and a second external indicia of the second stage,
wherein the finger flange defines a pointer, and
wherein rotating the collar relative to the finger flange to the rotation end location to transform the system to the primed/injection ready configuration moves the pointer on the finger flange from the first external indicia to the second external indicia on the finger flange.

26. The method of claim 23, wherein the finger flange comprises a proximally extending member having a pair of flex arms,
wherein the pair of flex arms provide an audible and/or tactile cue during rotation of the collar about the proximally extending member.

27. The method of claim 26, wherein the plunger rod comprises a barb,
wherein the bard interacts with a rib on the finger flange to limit proximal movement of the plunger rod relative to the finger flange.

28. The method of claim 26, wherein in the injected configuration, the portion of the injectable fluid ejected from the syringe interior is less than a total amount of the injectable fluid in the syringe interior.
